(19) United States
(12) Patent Application Publication
(10) Pub. No.: US 2022/0330837 A1
(43) Pub. Date: Oct. 20, 2022

(54) PROCESS AND DEVICE FOR DETERMINING A RESPIRATORY AND/OR CARDIOGENIC SIGNAL

(71) Applicant: Drägerwerk AG & Co. KGaA, Lübeck (DE)

(72) Inventors: Lorenz KAHL, Lübeck (DE); Philipp ROSTALSKI, Lübeck (DE); Eike PETERSEN, Lübeck (DE); Jan GRASSHOFF, Lübeck (DE)

(21) Appl. No.: 17/766,008

(22) PCT Filed: Aug. 26, 2020
(86) PCT No.: PCT/EP2020/073826

§ 371 Date: Apr. 1, 2022

(30) Foreign Application Priority Data
Oct. 2, 2019 (DE) 10 2019 006 866.1

(51) Int. Cl. A61B 5/0205; A61B 5/00

(52) U.S. Cl. CPC A61B 5/0205; A61B 5/7267; A61B 5/7253; A61B 5/7278

(57) ABSTRACT

A process and signal processing unit (5) determine a cardiogenic signal (Sig_{kar,est}) or a respiratory signal (Sig_{res,est}) from a sum signal (Sig_{Sum}), resulting from a superimposition of cardiac activity and breathing of a patient (P). A signal estimating unit (6), which yields a shape parameter as a value of a transmission channel parameter (LF), is generated during a training phase. A sample with a sample element per heartbeat is used. During a use phase, the transmission channel parameter is measured for each heartbeat, a shape parameter value is calculated by the application of the signal estimating unit and is used to calculate an estimated cardiogenic signal segment (Sig_{Hz,kar.LF}) or an estimated respiratory signal segment. The cardiogenic signal segments are combined into the cardiogenic signal or the respiratory signal segments are combined into the respiratory signal or the cardiogenic signal segments are subtracted from the sum signal.

### CROSS REFERENCE TO RELATED APPLICATIONS

This application is a United States National Phase Application of International Application PCT/EP2020/073826, filed Aug. 26, 2020, and claims the benefit of priority under 35 U.S.C. § 119 of German Application 10 2019 006 866.1, filed Oct. 2, 2019, the entire contents of which are incorporated herein by reference.

### TECHNICAL FIELD

The present invention pertains to a device for determining an estimate or representation for a respiratory and/or a cardiogenic signal from a signal determined by means of measured values, which determined signal results from a superimposition of the cardiac activity and the breathing and/or ventilation of a patient.

### TECHNICAL BACKGROUND

A “signal” shall be defined below as the curve or course in the time range or also in the frequency range of a directly or indirectly measurable indicator, which is variable over time and which is correlated with a physical variable. This physical variable is linked in this case with the cardiac activity and/or with the spontaneous (intrinsic) breathing of a patient and/or with a mechanical ventilation of the patient and is generated by at least one signal source in the body of the patient or by a ventilator. A “respiratory signal” is correlated with the spontaneous breathing and/or with a mechanical ventilation of the patient, and a “cardiogenic signal” is correlated with the cardiac activity of the patient.

The respiratory signal is, in particular, an indicator for the breathing pressure or an indicator for the flow of breathing air relative to the lungs of the patient, wherein this flow of breathing air is effected by the breathing pressure, and the breathing pressure and hence also the breathing flow is caused by the spontaneous breathing of the patient, by mechanical ventilation performed by a ventilator or by a superimposition of the spontaneous breathing and the mechanical ventilation. For example, the pressure in the airway, in the esophagus or in the stomach or an electromyogram may be used as an indicator for the breathing pressure, as a rule, as a pressure difference relative to the ambient pressure. The breathing air flow brings about a change over time in the lung filling level of the patient.

A possible application of the present invention is the control of a mechanical ventilator. This ventilator assists the spontaneous breathing of a patient. The ventilator shall perform ventilation strokes synchronized with the spontaneous breathing of the patient in order for the patient not to breathe against the ventilator. In order to synchronize the ventilator automatically with the spontaneous breathing of the patient, a respiratory signal is needed.

In many situations this respiratory signal cannot be measured in an isolated manner from the cardiogenic signal. Rather, only a sum signal can be obtained, which results from a superimposition of the breathing and/or ventilation and the cardiac activity of the patient. The influence of the cardiac activity on the sum signal shall at least approximately consequently be compensated by calculation in this application.

Conversely, it is frequently desirable to obtain and to use a cardiogenic signal, for example, an improved ECG signal. Only a sum signal, which results from a superimposition of the cardiac activity with the breathing and/or with the mechanical ventilation of the patient, is often available for this task as well. The influence of the breathing and/or ventilation on the sum signal shall be compensated at least approximately in this application. Even if the patient is fully sedated and is ventilated exclusively mechanically, i.e., the patient's intrinsic spontaneous breathing is greatly or even completely suppressed, the ventilation does influence the cariogenic signal.

In the first application, the respiratory signal is the wanted signal and the cardiogenic signal is an unwanted signal to be at least approximately compensated by calculation. In the second application, the cardiogenic signal is the wanted signal and the respiratory signal is the unwanted signal.

An approach to generating a wanted signal from a sum signal of a patient is described in M. Ungureanu and W. M. Wolf: Basic Aspects Concerning the Event-Synchronous Interference Canceller,” IEEE Transactions on Biomedical Engineering, Vol. 53, No. 11 (2006), pp. 2240-2247.

A process and a device for generating two data signals, wherein the first data signal describes an activity of a muscle responsible for inhalation and the second data signal describes an activity of a muscle relevant for exhalation, are described in DE 10 2015 015 296 A1. Two surface myography sensors detect two EMG signals. A cardiac signal component in the EMG signals is suppressed by calculation. In addition, the breathing activity of the patient is determined. A computer detects on the basis of the detected breathing activity when the patient is inhaling and when the patient is exhaling. A first separated signal and a second separated signal are determined on the basis of the two EMG signals.

A process for automatically controlling a ventilation system is described in DE 10 2007 062 214 83. A breathing activity signal uEMG(t) is recorded with electrodes on the surface of the chest in the process known from DE 10 2007 062 214 B3. To make electromyographic signals representing the breathing activity from the recorded electrode signals, the electrode signals must be subjected to a preprocessing; in particular, ECG signal components, which dominate the overall signal in terms of the signal height, must be removed. Filtering as well as an envelope detection may preferably be carried out for this purpose. The enveloping curve detection is preferably carried out by forming the absolute value or by raising to the second power and subsequent low-pass filtering of the electrode signals. Electromyographic signals, which represent the breathing activity and which can be used to control the ventilation drive of the ventilator, are obtained after this preprocessing, as this is described, e.g., in DE 10 2007 062 214 B3.

A medical sensor device 11 is described in DE 10 2009 035 018 A1. Electrodes 12 on the chest of a patient generate electrical signals, from which an electromyogram (sEMG) is generated. An array with an acceleration sensor 6 and with a microphone 7 generates a mechanomyogram (MMG). The measured signals contain an ECG component, which is suppressed by calculation by filtering. FIG. 10 shows an ECG signal 71 and a breathing signal 70. FIG. 11 shows an EMG/MMG signal 72 and a breathing signal 70.

WO 2005/096924 A1 describes a ventilation system (positive pressure ventilation device), which ventilates a patient as a function of EMG signals. Electrodes on the skin of the patient (skin surface electrodes) provide signals, in which the EMG signal being sought is superimposed by an ECG signal. The ECG component is removed from the measured signal by calculation, so that an EMG signal from which unneeded components have been removed (moving average electromyogram signal) is generated. This signal is displayed (displaying).

A process for generating signals of a fetus in the womb, especially the heartbeat activity of the fetus (fetal heart rate), is described in US 2007/0191728 A1. Electrodes 20, 21 and 22 on the abdomen of the pregnant mother measure a superimposition of ECG and EMG signals. The ECG signals are separated from the EMG signals by calculation, and the fetal signals are distinguished from the signals of the pregnant mother by calculation. EP 2371412 A1 shows a device for the mechanical ventilation or also anesthesia of a patient. An sEMG sensor 6 on the skin of the patient detects the electromyographic muscle activity of the breathing muscles of the patient.

U.S. Pat. No. 6,411,843 B1 describes a process and a device for obtaining a processed EMG signal (model EMG signal) from a measured signal, which is formed from a superimposition of an EMG signal and of an ECG signal of a patient. An envelope (first envelope signal) is calculated from the measured signal. In addition, heartbeat times are detected in the measured signal. The processed EMG signal is generated from the generated envelope and the detected heartbeat times. A first logical signal, in which the P wave, the QRS complex and the T wave are removed by calculation, and a second logical signal, in which the P wave, the QRS complex or the T wave are contained, are derived from the measured EMG signal. Furthermore, a first envelope is derived from the measured EMG signal. A modeled EMG signal is derived from the first envelope and from the first logical signal, on the one hand, and from a signal that depends on the second logical signal, on the other hand.

DE 10 2012 003 509 A1 describes a ventilation system with a control device and with a patient module. Electrodes of the patient module derive electrode signals from the surface of the chest of a patient. The control device suppresses in the electrode signals ECG components and derives first ECG signals. Data that represent the ECG are sent in the digital form to an ECG output, on the one hand, and are converted, on the other hand, into an analog signal, which is made available for display.

It is proposed in WO 2018001929 A1 that a first undesired signal component be reduced from a physiological signal by subtracting a model of the undesired signal from the physiological signal. A residual signal is obtained thereby. A filter unit reduces in the residual signal a second undesired signal by a band elimination filter (notch filter) generating a filtered signal. A gating is applied to the filtered signal

### SUMMARY

A basic object of the present invention is to provide a process and a signal processing unit, which determine an estimate or representation for a cardiogenic signal and/or for a respiratory signal from a sum signal, which sum signal is generated by means of measurements of a signal generated in the body of the patient and results from a superimposition of the cardiac activity of the patient to the spontaneous breathing and/or to the mechanical ventilation of the patient, better than do prior-art processes and signal processing units.

The object is accomplished by a process having the process features of the invention and by a signal processing unit having the device and system features of the invention. Advantageous embodiments are described herein. Advantageous embodiments of the process according to the present invention are also advantageous embodiments of the signal processing unit according to the present invention and vice versa.

According to the present invention, an estimated (representative) cardiogenic signal and/or an estimated (representative) respiratory signal are calculated. The calculated respiratory signal is correlated with the spontaneous breathing and/or with a mechanical ventilation and especially with the flow of breathing air relative to the lungs of the patient. This flow of breathing air may be brought about exclusively by the spontaneous breathing of the patient, exclusively mechanically by mechanical ventilation by means of a ventilator (e.g., the patient is fully sedated) or by the spontaneous breathing assisted by the mechanical ventilation. The calculated respiratory signal also contains a component, which is caused by the cardiac activity. This component is, however, as a rule, smaller than the component in the sum signal generated on the basis of the measurements. The determined cardiogenic signal is correspondingly an indicator for the cardiac activity of the patient. The cardiogenic signal contains a component, which is caused by the breathing or ventilation, and this component is smaller than the respiratory component in the sum signal.

The process according to the present invention comprises a training phase and a subsequent use phase and is carried out automatically with the use of the signal processing unit according to the present invention.

During the training phase the signal processing unit receives measured values from a sum signal sensor device comprising at least one sum signal sensor. The sum signal sensor device measures a signal, which is generated in the body of the patient. Optionally the signal processing unit receives measured values from the sum signal sensor device during the use phase as well.

The signal processing unit generates a sum signal during the training phase. This generated sum signal comprises a superimposition of the cardiac activity to the spontaneous breathing and/or to the mechanical ventilation of the patient. In order to generate the sum signal, the signal processing unit uses the respective time curve (temporal course) of measured values, which have been provided by the sum signal sensor device. Optionally the signal processing unit also generates the sum signal during the use phase.

The signal processing unit detects during the training phase a plurality of heartbeats, which the patient has performed during the training phase, preferably each heartbeat. The signal processing unit generates a sample, which comprises a plurality of sample elements. Each sample element of the sample pertains to a respective heartbeat detected during the training phase.

To generate a sample element for a heartbeat, the signal processing unit carries out the following steps:
The signal processing unit determines a segment of the sum signal, which segment belongs to this heartbeat.
The signal processing unit determines at least one shape parameter value. The or each parameter value is a value which a shape parameter assumes at this heartbeat. The shape parameter or each shape parameter influences the curve of the cardiogenic signal and/or of the respiratory signal. In other words, the curve of the cardiogenic signal and/or the curve of the respiratory signal depends on the shape parameter value or each shape parameter value. If another value is assigned to the shape parameter or to a shape parameter, the cardiogenic signal and/or the respiratory signal changes its shape in a graphic presentation.
The signal processing unit determines at least one value for a predefined first transmission channel parameter and optionally at least one value for another predefined transmission channel parameter. The channel parameter takes this value during the heartbeat. The first and the optional additional transmission channel parameters are correlated with a respective effect, which an anthropological variable has on a transmission channel. This transmission channel leads from a signal source in the body of the patient, especially from the breathing muscles and/or the heart muscle, to the sum signal sensor device. The anthropological variable is generated in the body of the patient and is linked especially with the spontaneous breathing and/or with the mechanical ventilation of the patient or with irregularities in the cardiac activity of the patient.
The step of the signal processing unit determining the value for the first transmission channel parameter comprises in a first alternative the step of the signal processing unit receiving this value. The value was measured at the heartbeat by an additional sensor at the patient and transmitted to the signal processing unit. In a second alternative, the signal processing unit calculates the value for the first transmission channel parameter, wherein it analyzes the sum signal.
The signal processing unit generates the sample element for this heartbeat such that the sample element comprises the following: the respective value of the shape parameter or each shape parameter that has been calculated for this heartbeat, as well as the value or a value of the first transmission channel parameter, which was determined, i.e., received or calculated, at this heartbeat.

The signal processing unit generates in the training phase a signal estimating unit (signal representation unit). The generated signal estimating unit yields the shape parameter or each shape parameter (at least one shape parameter) as a function of the first transmission channel parameter and optionally as a function of at least one additional transmission channel parameter. The signal processing unit uses the sample with the sample elements for the generation.

In the use phase, the signal processing unit detects at least one heartbeat, which the patient carries out in the course of the use phase. The signal processing unit preferably detects each heartbeat in the use phase or at least in a time period of the use phase.

The signal processing unit carries out the following steps during the use phase for at least one detected heartbeat and preferably for each detected heartbeat:
The signal processing unit detects a characteristic time and/or a time period of the heartbeat.
The signal processing unit determines a value that the first transmission channel parameter assumes at this heartbeat. In a first alternative of determining the value, the signal processing unit receives this value from the or from the additional sensor, which has measured the first transmission channel parameter during the heartbeat. In a second alternative, the signal processing unit calculates this value by generating and analyzing the sum signal during the use phase as well.
The signal processing unit calculates for the shape parameter or for each shape parameter a respective value that the shape parameter assumes at this heartbeat. To calculate the shape parameter or each shape parameter at this heartbeat, the signal processing unit applies the generated signal estimating unit to the determined value of the first transmission channel parameter and optionally to the respective determined value of each additional transmission channel parameter.
The signal processing unit calculates an estimated cardiogenic signal segment and/or an estimated respiratory signal segment for this heartbeat. This signal segment is correlated with the cardiac activity and with the spontaneous breathing and/or mechanical ventilation of the patient in the course of the heartbeat and thus describes approximately the cardiogenic signal and/or the respiratory signal in the course of this heartbeat. To calculate the estimated signal segment, the signal processing unit uses the calculated shape parameter value or each calculated shape parameter value.

In a first alternative of the present invention, the signal processing unit determines in the use phase the estimated cardiogenic signal. For doing so, it combines the estimated cardiogenic signal segments for the heartbeats detected during the use phase to the estimated cardiogenic signal. In a second alternative of the present invention, the signal processing unit determines during the use phase the estimated respiratory signal. For doing so, it combines the estimated respiratory signal segments for the heartbeats detected during the use phase to the estimated respiratory signal. In a third alternative of the present invention, the signal processing unit likewise determines during the use phase the estimated respiratory signal, but, contrary to the second alternative, it does so by a compensation by calculation. This determination by compensation comprises the following steps:
The signal processing unit generates a sum signal during the use phase as well. It uses for this received measured values, which the sum signal sensor device, comprising at least one sum signal sensor, has measured.
The signal processing unit compensates by calculation the respective influence of at least one heartbeat, which has been detected during the use phase, on the sum signal generated during the use phase. The signal processing unit preferably compensates the respective influence of each detected heartbeat. To compensate the influence of a heartbeat, the signal processing unit uses the estimated cardiogenic signal segment for this heartbeat. It preferably subtracts this estimated cardiogenic signal segment from the sum signal.

Thanks to the present invention, it is not necessary to generate the respiratory signal or the cardiogenic signal by direct measurement. This is, as a rule, not possible at all, or even though it is possible, it is not desired, e.g., because a sensor and/or a maneuver needed therefor would stress the patient too strong during the operation of the ventilator. A sum signal is rather generated according to the present invention from the measured values of the sum signal sensor device, and the respiratory signal and/or the cardiogenic signal is determined by calculation using this sum signal.

According to the present invention, a signal estimating unit is generated automatically and a sample with a plurality of sample elements, which was generated during the training phase, is used for this generation. Since a sample is generated empirically and then used, no analytical model is needed; in particular, no model that analytically describes the influence of the cardiac activity or of the breathing/ventilation is needed. Such a model often cannot be set up at all or it can be set up and validated and adapted to a patient at an unacceptably great effort only. However, the present invention can be used in a plurality of configurations combined with an analytical model.

According to the present invention, this sample is generated with the use of measured values that are measured during the training phase at that patient for whom the steps of the subsequent use phase are carried out as well. The present invention therefore avoids errors that would appear, as a rule, if measurements were carried out during the training phase on at least one patient and the results of the training phase were applied in the use phase to another patient. Such errors would often also occur if measurements were carried out during the training phase on a plurality of patients and averaging was performed over the measurements.

Since according to the present invention a plurality of sample elements are used in order to generate the signal estimating unit, the influence of freak values is removed to a certain extent by averaging.

The same sum signal sensor device can be used in both the training phase and in the use phase. The use of different sensor devices during the two phases, which is avoided according to the present invention, could cause additional errors. The present invention avoids this possible source of error.

According to the present invention, the signal estimating unit, which is generated during the training phase, yields a respective estimated signal segment for at least one and preferably for each heartbeat detected during the use phase. The provided estimated signal segment may differ from one heartbeat to the next. The present invention takes into consideration the following circumstance: The anthropological variable, especially the spontaneous breathing and/or the mechanical ventilation of the patient, influences the respective transmission channel guiding from nerves and/or muscles, which elicit the cardiac activity and/or the spontaneous breathing, to the sum signal sensor device. The spontaneous breathing therefore acts additionally as a disturbance signal on the cardiogenic signal and hence also on the sum signal. The influence of the spontaneous breathing varies, as a rule, from one heartbeat to the next. A mechanical ventilation of the patient also influences such a transmission channel, and this influence may vary from one heartbeat to the next. The first transmission channel parameter, which is taken into account according to the present invention, correlates with the effect that the spontaneous breathing or mechanical ventilation or another anthropological variable has on the transmission channel to the sum signal sensor device, and can be measured. This transmission channel is located completely or at least partially in the body of the patient. Due to the first transmission channel parameter being measured and due to the measured transmission parameter value being analyzed, the effect of breathing and/or ventilation or another anthropological variable on the transmission channel and hence on the cardiogenic signal can at least approximately be taken into account.

In other applications, the anthropological variable may correlate with the cardiac activity of the patient and act as a disturbance signal on the respiratory signal and hence on the sum signal. The present invention makes it possible to compensate the influence of this disturbance variable by calculation in these applications as well.

The signal estimating unit provides in the use phase a respective estimated signal segment each for at least one and preferably for each detected heartbeat. This estimated signal segment pertains to the time period of an individual detected heartbeat. According to the present invention, the estimated signal segment for a heartbeat depends on the value or on at least one value for the first transmission channel parameter that was measured during this heartbeat. The estimated signal segment calculated by the signal estimating unit does consequently take into consideration at least approximately the effect of the anthropological variable, especially the effect of the spontaneous breathing or mechanical ventilation or also of the cardiac activity on the transmission channel during this heartbeat.

If, by contrast, the same estimated or predefined signal segment, for example, a predefined standard signal segment (e.g., a so-called ECG template), were used for each heartbeat, the varying influence especially of breathing or ventilation on the or one transmission channel could not be taken into account at all or it would be able to be taken into account at least only to a markedly lesser extent.

The estimated signal segment for a heartbeat, which is calculated during the use phase, depends on the value or on at least one value for the first transmission channel parameter that was measured in the use phase during this heartbeat. This estimated signal segment is therefore adapted to the anthropological variable, more precisely, adapted to the influence that the anthropological variable in the body of the patient has on the transmission channel from at least one muscle or from another signal source in the body of the patient to the sum signal sensor device used during this heartbeat. The signal source, in which a transmission channel to a sum signal sensor device begins, is, e.g., a heart muscle or a muscle of the respiratory system.

According to the present invention, the signal estimating unit yields during the use phase an estimated signal segment for the time period of this heartbeat. This signal estimating unit is generated during the training phase automatically by means of a sample, wherein each sample element of this sample comprises at least one transmission channel parameter value and at least one associated shape parameter value.

In a first alternative of the present invention the estimated signal segment describes a segment of the estimated cardiogenic signal in the course of this heartbeat. The signal processing unit combines these segments during the use phase to the cardiogenic signal. In another alternative, the signal processing unit uses at least one segment for a heartbeat (estimated cardiogenic signal) in order to compensate the influence of the cardiac activity during this heartbeat on the sum signal by calculation, especially by the segment being subtracted from the sum signal. In yet another alternative, the estimated signal segment describes a segment of the estimated respiratory signal in the course of this heartbeat. In one embodiment, the signal processing combines these segments to the respiratory signal.

In one embodiment, the entirety of the shape parameter values of a sample element for a heartbeat specifies a segment of the respiratory or cardiogenic signal to be determined in the course of this heartbeat. The shape parameter values are, for example, support points of a reference signal segment in the course of a heartbeat.

In another embodiment, a standard reference signal segment is predefined, which segment is valid for each heartbeat and preferably for each patient and depends on at least one shape parameter and preferably describes the cardiac activity. The shape parameter value or the entirety of the shape parameter values together with this standard reference signal segment specify the segment of the sum signal in the time course of the heartbeat during the training phase and the estimated signal segment during the use phase. Preferably the estimated signal segment describes in this application a segment of the cardiogenic signal.

In one embodiment, a change rule is predefined, which vile depends on the shape parameter or on at least one shape parameter. During the use phase the signal estimating unit yields at least one respective shape parameter value for each segment and uses this value for the change rule. The signal processing unit determines for each heartbeat the segment of the sum signal, which segment belongs to this heartbeat. The signal estimating unit applies the change rule parameterized for this heartbeat to a determined sum signal segment. As a result, the signal estimating unit yields the estimated signal segment for this heartbeat. The estimated signal segment can describe a segment of the respiratory signal or of the cardiogenic signal to be determined.

In another embodiment, the shape parameter value or the entirety of the shape parameter values specify a calculation rule wherein the rule is adapted to calculate the estimated signal segment for the heartbeat in question from the segment of the sum signal for a heartbeat, which segment was determined during the use phase.

In one embodiment, a computer-accessible library with a plurality of reference signal segments is generated during the training phase, and each reference signal segment pertains to a class of possible values of the first transmission channel parameter and optionally of at least one additional transmission channel parameter and describes a segment of the estimated cardiogenic or respiratory signal during a heartbeat. Each reference signal segment is generated with the use of at least one and preferably a plurality of sample elements, whose parameter values fall within this class, wherein corresponding segments of the sum signal are combined in a suitable manner to the reference signal segment. The value or at least one value of the first transmission channel parameter and optionally a respective value of each additional transmission channel parameter is measured for a detected heartbeat during this heartbeat during the use phase. Depending on the measured parameter value, at least one reference signal segment is selected in the library, and the estimated signal segment is generated depending on the reference signal segment or each selected reference signal segment.

For example, the two reference signal segments that belong to the two parameter values that are located adjacent to the measured value of the first transmission channel parameter are selected, and the estimated signal segment is generated as a weighted mean value over these two selected reference signal segments. The weighting factors are calculated, e.g., such that the estimated signal segment is an interpolation between the two reference signals.

The embodiment with the library causes an estimated signal segment to be calculated rapidly during the use phase for a detected heartbeat and relatively little storage space to be needed.

In one embodiment, a plurality of classes pertain to partial areas of a regular area of the first transmission channel parameter or of an additional transmission channel parameter and to at least one additional class of “freak values,” which occur in unusual situations, for example, when the patient is coughing or has a muscle spasm or exerts himself excessively or the patient's heartbeat shows a spontaneous irregularity.

In one embodiment, a plurality of transmission channel parameters are predefined and taken into consideration, and these transmission channel parameters influence the transmission channel or at least one respective transmission channel to the sum signal sensor device. The signal estimating unit is generated during the training phase such that the signal estimating unit yields an estimated signal segment in the course of a heartbeat as a function of a plurality of transmission channel parameters. This embodiment makes it possible to take into consideration simultaneously a plurality of different influential factors on the same transmission channel. It is possible, but thanks to the present invention not necessary to determine such parameters, which are dependent on one another in advance by calculation. This may require a large amount of computing time and/or be time-consuming.

The training phase comprises a plurality of heartbeats, preferably between 20 heartbeats and 60 heartbeats. The use phase preferably begins immediately after the end of the training phase.

In one embodiment, the training phase ends after a predefined number of heartbeats and/or after a predefined time period. It is also possible that the training phase ends as soon as a sufficient number of different values have been measured for the first transmission channel parameter or for each transmission channel parameter.

According to the present invention, a respective characteristic time and/or a time period of a heartbeat is measured during the use phase. In one embodiment, the sum signal sensor device, comprising at least one sum signal sensor, yields an electrical signal, and the fact is utilized that an electrical signal, which is caused by an individual heartbeat, typically has a curve that comprises a P wave, a QRS wave and a T wave. These waves and the corresponding peaks can also be determined in the sum signal, because the percentage of the cardiogenic signal between the P wave and the T wave is several times greater than the percentage of the respiratory signal. This designation P through T has become established in the literature. The Q peak, the R peak or the S peak of this heartbeat, especially the R peak, is preferably used as the characteristic time of a heartbeat. In another embodiment, a respective heartbeat time period is determined during the use phase for each heartbeat, and the heartbeat takes place during this time period and/or the determined time period comprises this heartbeat. The heartbeat time period extends, for example, from the P wave to the T wave. The heartbeat time period is determined, for example, by an analysis of the sum signal. At least when the sum signal is determined by means of measured values of electrical sensors, the influence of the heartbeats in the sum signal is many times greater than the influence of the breathing activity. The time in this determined heartbeat time period at which the sum signal assumes a maximum or a minimum is preferably detected as the characteristic time of the heartbeat.

In an alternative of the present invention, the influence of at least one detected heartbeat, preferably of each detected heartbeat, on the sum signal is compensated by calculation when a respiratory signal shall be determined. Different embodiments of how this compensation can be carried out are possible. In one embodiment a heartbeat time period is determined for a detected heartbeat. The sum signal and the characteristic heartbeat time period are used for this. For example, the heartbeat time period covers the P wave, the QRS wave and the T wave. The estimated signal segment for the heartbeat is subtracted from the sum signal in the heartbeat time period, or the estimated signal segment multiplied by a factor and/or shifted by a time delay is subtracted.

According to the present invention, at least one value, which the first transmission channel parameter assumes at this heartbeat, is measured for each heartbeat. Optionally a value of at least one additional transmission channel parameter is measured at this heartbeat. The term “value” may designate a single number, i.e., a scalar, or also a vector. For example, the position of a sum signal sensor relative to the heart or to another reference point in the body of the patient may be used as a transmission channel parameter. This relative position depends on the current lung filling level. Each value of this transmission channel parameter is preferably a vector with three components, for example, in a three-dimensional cartesian coordinate system.

In one embodiment the sum signal can be generated by means of passively operating measuring electrodes, which electrodes are positioned at or in the body of the patient and provide a respective electrical measured value each (especially surface electromyogram or electromyogram in the body, e.g., in the esophagus or in the stomach). Each measured electrical value depends on the current activity of the diaphragmatic muscles as well as on the activity of the auxiliary muscles and optionally on the mechanical ventilation of the patient. The measured values of the measuring electrode can be analyzed, which leads to an electrical sum signal.

In many cases, a prediction can be made about the patient on the basis of a respiratory signal, which was determined according to the present invention from an electrical sum signal, better than with other methods. For example, the heart rate can better be predicted based on a cardiogenic signal, which was determined according to the present invention from the electrical sum signal.

In one embodiment, an electrical impedance tomography belt (EIT belt) is used as a sum signal sensor device and/or as a sensor for a transmission channel parameter. Such an EIT belt is applied on the skin of the patient and it comprises a plurality of signal units, which can be operated alternatingly as signal source or signal receiver. At each time exactly one signal unit is a signal source, and the other signal units are signal receivers. The signal source generates a high-frequency signal, preferably in the range of several kHz, which is harmless for the patient and penetrates into the body of the patient. The EIT belt measures the respective electrical impedance in the body of the patient between the signal source and a signal receiver. The electrical impedance in a body part filled with air, especially in the lungs, is several times higher than the electrical impedance in a tissue, which is filled with a salt-containing and therefore electrically conductive solution.

The EIT belt thus generates an image of the lungs in the body of the patient, which is variable over time. If the lung filling level of the patient is the transmission channel parameter or a transmission channel parameter, the signal processing unit is capable of determining the current lung filling level from the image of the lungs, e.g., by image processing. It is also possible that the signal processing unit uses the image of the lungs, which is variable over time, as a sum signal.

In one embodiment, the image of the lungs is divided into a plurality of areas, wherein each area shows a region of the lungs. For example, the image is divided into four quadrants or into a plurality of pixels. Each image area is used as a respective sum signal. By analyzing this sum signal or these sum signals, the signal processing unit is capable of detecting the heartbeats. It is also possible that the signal processing unit receives measured values from another sensor, which detects the heartbeat times and/or heartbeat time periods.

The measured electrical value, which is brought about by the heart muscle, is several times higher than the measured value that is brought about by the breathing muscles. The cardiac activity causes voltages in the mV range, and the breathing activity causes voltages in the microV range. The higher voltages of the cardiac activity occur essentially only from time to time, namely, from time to time in the course of a heartbeat, and not during the rest of the time nor between the heartbeats. It is especially because of this that it is possible to obtain the respiratory signal from the sum signal.

According to the present invention, the respective value, which the or each shape parameter assumes at the heartbeat in question, is determined for each heartbeat. The segment of the sum signal belonging to this heartbeat is preferably used for this determination. Especially if the sum signal is an electrical signal, the sum signal in a heartbeat is essentially equal to the cardiogenic signal. The influence of the respiratory signal is often averaged over a plurality of sample elements when the signal estimating unit is generated.

It is also possible to obtain the sum signal by means of at least one pneumatic sensor, in which case the pneumatic sensor measures, for example, an indicator for the flow of gas into or out of the lungs of the patient and/or the airway pressure. This flow is measured, e.g., at a ventilator which is connected to the patient, or at the mouth of the patient. For example, the volume flow and the achieved ventilation pressure are measured in a fluid connection between the patient and the ventilator. A time delay between the lungs of the patient and the connection ventilator is predefined or estimated, and the time delay is used to correct measurements that are carried out at the ventilator in terms of time and to compensate the delay by calculation in the process.

In one embodiment, the sum signal sensor device comprises a probe or a balloon or a catheter, which is inserted into the body of the patient, for example, into the esophagus, and an electrical or pneumatic transducer. It is also possible to measure the breathing muscles by means of a sensor for a mechanomyogram or vibromyogram. In one embodiment, at least one catheter, which measures the esophageal pressure or the gastric pressure, is used as a sum signal sensor of the sum signal sensor device.

In another embodiment the sum signal sensor device comprises an image recording device, which is directed towards the patient. An imaging method is applied to the signals from the image recording device. This embodiment eliminates the need to position the sum signal sensor at or even in the patient. Rather, a spatial distance between the patient and the sum signal sensor remains. This embodiment leads to a greater tolerance in case of deviations between a desired position and an actual position of a sum signal sensor relative to the patient.

It is also possible to combine different types of sensors with one another. The sum signal is generated in this embodiment from measured values of different sensors.

According to the present invention, the signal processing unit receives measured values from the sum signal sensor or from at least one sum signal sensor. The measured values are preferably processed, for example, amplified and/or smoothed, and/or disturbing influences are filtered out of the measured values. In addition, analog measured values are converted into digital measured values. If a measuring electrode is positioned on the skin of the patient and is used as the or one sum signal sensor, electrochemical effects, which develop due to the contact between the measuring electrode and the skin, especially between the silver of the electrode and sweat on the skin, are preferably compensated by calculation (baseline removing, baseline filtering), and other potential differences are compensated. The signal processing unit generates the sum signal from the measured values processed in this manner and uses especially the processed measured values as the sum signal.

According to the present invention, at least one value per heartbeat of the first transmission channel parameter is measured. This measured transmission channel parameter is correlated with at least one anthropological variable, which influences a transmission channel from a signal source in the body of the patient to the sum signal sensor or to at least one sum signal sensor. In one embodiment, the anthropological variable or an anthropological variable is the current geometry of the body of the patient. This body geometry depends in many cases on the current lung filling level of the patient. The first transmission channel parameter is thus correlated with the lung filling level of the patient.

In one embodiment, a mechanical or pneumatic or optical sensor measures an indicator for the body geometry, for example, the flow of breathing air into the lungs or out of the lungs or the body circumference of the patient in such a measurement position in which the body circumference varies with the lung filling level. An optical sensor comprises especially an image recording device and an image analysis unit, which employs an imaging method. The variable body geometry influences the transmission channel from the heart or from a part of the breathing muscles to the sum signal sensor or to at least one sum signal sensor, for example, because the distance varies.

In one embodiment, the current posture or body position of the patient is used as the transmission channel parameter or as a transmission channel parameter, for example, the position of the patient in a bed or whether the upper body of the patient is upright or curved. The posture also influences the transmission channel.

In one embodiment, the anthropological variable causes the time interval between two consecutive heartbeats to vary and has, for example, a periodicity covering at least two heartbeats or is irregular. This interval is an indicator indicating how rapidly the heart muscle recovers after a heartbeat. Or else the time interval between two peaks of the sum signal is influenced by the anthropological variable, and the peaks are reached in the course of a heartbeat. The anthropological variable is, for example, the posture of the patient or even an irregularity in the cardiac activity. The time interval between two consecutive heartbeats or the time interval between two peaks in the course of the same heartbeat, e.g., the amplitude of this heartbeat, is used as the first transmission channel parameter or as an additional transmission channel parameter. The signal estimating unit yields the estimated signal segment as a function of the heartbeat distance. This embodiment requires no additional sensor for the first transmission channel parameter. The measured values of the sum signal sensor or the measured values of the array of sum signal sensors rather yield both the sum signal and the values of the first transmission channel parameter. Or else the value of the first transmission channel parameter is calculated by analyzing the sum signal.

In one embodiment, existing knowledge about the signal being sought in the course of a heartbeat is used. This existing knowledge was gained, for example, by means of a plurality of samples on a plurality of patients. In one embodiment the existing knowledge, which is caused by the cardiac activity in the course of a heartbeat and which depends on the shape parameter or at least one shape parameter, is predefined in the form of a standard reference signal segment for the process according to the present invention. During the training phase the signal processing unit generates a signal estimating unit, which yields the shape parameter or each shape parameter of the standard reference signal segment in the form of the used parameter or each used transmission channel parameter. During the use phase the signal processing unit applies for each detected heartbeat the signal estimating unit to the respective value of the measured transmission channel parameter or of each measured transmission channel parameter, which yields a respective value of each shape parameter. Using these shape parameter values, the signal processing unit adapts the predefined standard reference signal segment anew for each detected heartbeat, for example, by the signal processing unit introducing the shape parameter values into the standard reference signal segment. The standard reference segment adapted in this manner acts as the estimated signal segment for this heartbeat, or the estimated signal segment depends on the adapted standard reference signal segment in another manner. The shape parameter or a shape parameter may be, e.g., a time shift, a compression factor/stretching factor along the time axis or a signal amplification factor. The shape parameter or a shape parameter may influence the entire standard reference signal segment or also only at least one defined segment of the standard reference signal segment, e.g., segments with a great slope or segments with a small slope.

This embodiment with the standard reference signal segment, which is valid for each heartbeat and is parameterized, saves computing time and/or storage space in many cases. In order to specify a segment of a sum signal, substantially more points are needed, as a rule, than there are shape parameters. A maximum of five, at times even only three shape parameters are often sufficient.

In one embodiment a single such standard reference signal segment is used. In another embodiment, the value range of the first transmission channel parameter and/or of an additional transmission channel parameter is divided into classes in advance. A respective standard reference signal segment, which depends on the shape parameter or a shape parameter, is assigned to each class. During the training phase the signal processing unit generates a respective signal estimating unit each for each class and hence for each standard reference signal segment. During the use phase the signal processing unit decides for the detected heartbeat into which class the value of the first transmission channel parameter or of an additional transmission channel parameter, which value was measured during this heartbeat, falls. It selects the associated standard reference signal segment as well as the adapted signal estimating unit and fits the selected standard reference signal segment by applying the selected signal estimating unit.

In one embodiment, the signal processing unit carries out all process steps in the time range. In another embodiment, the signal processing unit transforms during the training phase for each heartbeat a segment of the sum signal belonging to this heartbeat from the time range into the frequency range. The generated signal estimating unit yields an estimated signal segment in the frequency range as a function of the first transmission channel parameter and in one embodiment additionally as a function of another transmission channel parameter. During the use phase the signal processing unit calculates for at least one detected heartbeat an estimated signal segment in the frequency range, transforms this segment into an estimated signal segment in the time range and uses the estimated signal segment in the time range in the manner according to the present invention. It is also possible that a respiratory or cardiogenic signal is generated and used in the frequency range from the sum signal generated in the time range by applying the process according to the present invention in the use phase.

The embodiment of transforming a segment of the sum signal during the training phase makes it possible to apply defined signal processing processes in the frequency range, for example, in order to remove disturbance signals with defined frequencies and to generate sample elements from cleaned-up segments of the sum signal in the frequency range. For example, the signal processing unit applies a low-pass filter, a high-pass filter and/or another band pass filter, removes frequencies in certain ranges, for example, in the range of the line voltage (50 Hz in Germany), or applies wavelet denoising or empirical-mode decomposition-based denoising. In one embodiment, at least one first frequency range is predefined, and in a preferred embodiment a plurality of preferably disjunct first frequency ranges are predefined. The signal processing unit generates an overall sum signal. The signal processing unit determines for each predefined first frequency range a respective signal component each, which is in this first frequency range. The signal processing unit determines, furthermore, a respective respiratory signal component and/or a cardiogenic signal component for the first frequency range or for each first frequency range and/or a cardiogenic signal component. The signal processing unit applies here the process according to the present invention for the first frequency range or for each first frequency range repeatedly, and the signal processing unit uses the signal component in this first frequency range as the sum signal. The signal processing unit subsequently determines the respiratory signal and uses for this determination the respiratory signal component or each respiratory signal component that is in the first frequency range or in a first frequency range and was determined by the use of the process according to the present invention. For example, it adds these respiratory signal components. Or else, the signal processing unit determines the cardiogenic signal and uses for this the cardiogenic signal component or each cardiogenic signal component in the first frequency range or in a first frequency range.

According to the present invention, the value which the respective transmission channel parameter assumes at a heartbeat is measured for the first transmission channel parameter and optionally for at least one additional transmission channel parameter. The signal processing unit receives these transmission channel parameter values. In one embodiment, a respective value each is measured for each transmission channel parameter and for each heartbeat. In another embodiment a split-up of the heartbeat period into at least two heartbeat time period phases, which split-up is valid for each heartbeat, is predefined. For example, a split-up into a first phase with the P-wave or P peak, into a second phase with the QRS-wave or QRS peak and into a third phase with the T-wave or T peak is predefined.

In this embodiment the signal processing unit receives for each detected heartbeat and for each transmission channel parameter a respective value per heartbeat time period phase of this heartbeat. During the training phase the signal processing unit generates a respective sample element each for each heartbeat time period phase of each detected heartbeat. If the training phase comprises, for example, 50 heartbeats and three heartbeat time period phases are predefined, the signal processing unit generates 50 sample elements per phase, i.e., a total of 50×3=150 sample elements.

During the use phase the signal processing unit calculates a respective shape parameter or—in case of a plurality of shape parameters—a set of shape parameter values for one and preferably for each detected heartbeat and for each heartbeat time period phase of this heartbeat. In case of ten shape parameters and three predefined heartbeat time period phases, these equal 10×3=30 shape parameter values per detected heartbeat. The signal processing unit calculates the estimated signal segment for this heartbeat with the use of the shape parameter values for the heartbeat time period phases, i.e., for example, of the 30 shape parameter values.

Preferably the signal processing unit generates in the training phase a respective signal phase estimating unit for each heartbeat time period phase. This signal phase estimating unit is valid for this heartbeat time period phase and yields, just like the signal estimating unit, the shape parameter or each shape parameter as a function of the transmission channel parameter or each transmission channel parameter. To generate this signal phase estimating unit. the signal processing unit uses the sample elements that belong to this heartbeat time period phase.

In this embodiment, the signal processing unit applies for each detected heartbeat the signal phase estimating unit for a heartbeat time period phase to the transmission channel parameter or to each transmission channel parameter that was obtained in this heartbeat time period phase of this heartbeat. As a result, a signal segment that describes the respiratory or cardiogenic signal in this heartbeat time period phase of this heartbeat is used. The signal processing unit generates the estimated signal segment for the heartbeat with the use of all signal segments for the phases of this heartbeat. For example, the signal processing unit combines the signal segments for the heartbeat time period phases to the estimated signal segment.

In one embodiment, the signal estimating unit, which the signal processing unit has generated during the training phase, is used unchanged during the entire use phase. In a preferred embodiment, the signal estimating unit is, by contrast, adapted during the use phase at least once, preferably continually, to the measured values obtained so far during the use phase. In this preferred embodiment, the signal processing unit generates the sum signal also during the use phase. In addition, the signal processing unit generates during the use phase at least one additional sample element, which pertains to a heartbeat detected in the use phase, preferably one additional sample element each for each heartbeat detected during the use phase. The signal estimating unit generated during the training phase is adapted during the use phase at least once with the use of the sample element or of another sample element. The signal estimating unit is preferably adapted continually to all further sample elements generated up to that time during the use phase. For example, a signal estimating unit is generated again repeatedly, doing so by means of a sample from the sample elements of the training phase and from the sample elements or at least some sample elements generated up to that point during the use phase.

In other words, the training phase acts in this embodiment as a starting phase for the generation of the signal estimating unit, and the use phase overlaps an improvement phase or adaptation phase for the signal estimating unit.

In particular, this embodiment makes it possible to take into consideration at least approximately the following influential factors in the course of the use phase:
The position of a sum signal sensor relative to the patient changes. For example, a measuring electrode changes its position on the skin of the patient.
The patient is moving, for example, turning in a bed or is changing the patient's posture.
The anthropological size changes its influence on the transmission channel in another manner, for example, because the patient is coughing or is exerting himself physically in another manner.
An operating parameter, e.g., the PEEP pressure (positive end-expiratory pressure), is changed during the mechanical ventilation of the patient.

In one embodiment, a respiratory signal is determined during the use phase. This respiratory signal can be used, for example, for the following applications:
The patient is ventilated by means of a mechanical ventilator. This ventilator carries out ventilation strokes. Each ventilation stroke is triggered automatically and depending on the respiratory signal determined so far during the use phase, preferably with the control goal of having the ventilation strokes carried out synchronously with the intrinsic breathing activity of the patient.
A ventilator signal is measured. This signal describes the flow of gas between the ventilator and the patient, wherein this gas flow is brought about by the ventilation strokes, which the ventilator carries out. This ventilator signal is compared to the respiratory signal. In case of a deviation above a threshold, asynchrony is detected, i.e., especially a phase shift between the ventilation strokes of the ventilator and the breathing activity of the patient. A corresponding alarm is outputted. In response to the output of this alarm, a user can set an operating parameter of the processing device (of the ventilator) at a different set point. Or else the signal processing unit causes the ventilator parameter to be set automatically at a different set point.
An electrical sum signal is generated by means of at least one electrical sum signal sensor. A mechanical or pneumatic sum signal is generated by means of at least one mechanical or pneumatic sum signal sensor. Due to the invention being applied twice, an electrical respiratory signal and a mechanical or pneumatic respiratory signal are generated.
From these two respiratory signals a functional relationship between the mechanical or pneumatic activity of the breathing muscles, which is measured by the set of mechanical or pneumatic sum signal sensors, and the measured values of the electrical sum signal sensor or sensors is derived. In particular, a coupling factor is derived, which describes the neuromechanical efficiency, i.e., it describes how well electrical signals are converted into muscular activity in the body of the patient.
This functional relationship can be used, on the one hand, to determine whether the respiratory muscles of the patient convert intrinsic electrical signals of the body correctly into breathing strokes or not. Furthermore, an electrical signal can be converted into a mechanical or pneumatic signal and vice versa, so that only one type of sum signal sensors will be needed later.
The current state of the breathing muscles of the patient is determined, for example, concerning the pressure generated or the force applied by the breathing muscles. The signal processing unit preferably determines the amplitude and/or the time curve of the amplitude of the respiratory signal determined and compares this amplitude to a predefined lower limit.
It is detected, in particular, whether and if yes, to what extent the breathing muscles of the patient are fatigued (fatigue detection).
Due to a suitable method of signal processing being applied, unusual contractions of the breathing muscles, for example, spasms or cough or hiccup, can automatically be detected.
In one embodiment, the ventilator is set depending on the detected fatigue of the breathing muscles and the setting is changed when needed.
The breathing muscles of the patient are trained in order to make it possible to end the mechanical ventilation of the patient as rapidly as possible. Both an underchallenge and an overchallenge of the breathing muscles must be avoided here. The respiratory signal is used to train the breathing muscles and to observe this boundary condition in the process.

The cardiogenic signal generated according to the present invention can be used instead of a conventionally determined ECG signal, and the same measuring electrodes can continue to be used. The cardiogenic signal approximately compensates the influence of the anthropological variable or at least one anthropological variable, especially that of the breathing activity on the measured signal.

The present invention will be described below on the basis of an exemplary embodiment. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a plurality of measuring electrodes positioned on a patient and a plurality of additional sensors positioned on and above the patient, wherein the patient is being ventilated by a mechanical ventilator;

FIG. 2 is a schematic view showing the determination of the respiratory and cardiogenic signals from the sum signal;

FIG. 3 is a schematic view showing how a cardiogenic signal is composed from estimated signal segments for individual heartbeats;

FIG. 4 is a schematic view showing how the influence of a transmission channel parameter is taken into consideration in the device according to FIG. 2;

FIG. 5 is a schematic view of an embodiment showing how two transmission channel parameters are taken into consideration in the device according to FIG. 4;

FIG. 6 is a schematic view showing in an exemplary manner several steps that are carried out during the use phase;

FIG. 7 is a graph showing an electrical cardiogenic signal in the course of a single heartbeat;

FIG. 8 is a graph showing as an example how sample elements, and from these a signal estimating unit, are generated and how estimated signal segments are generated and are combined into the estimated cardiogenic signal;

FIG. 9 is a graph showing a variant of the graph shown in FIG. 8, in which the filling level of the lungs is determined by a pneumatic sensor;

FIG. 10 is a view showing how during the training phase in the variant according to FIG. 9 the respective estimated signal segment of a class is formed in the course of a heartbeat from the segments that belong to a heartbeat each and to a lung filling level;

FIG. 11 is a graph showing another variant of the graph shown in FIG. 8, in which the lung filling level is determined by an analysis of image sequences;

FIG. 12 is a graph showing another variant, in which only signals in a defined frequency range are taken into consideration;

FIG. 13 is a view showing how four shape parameter values (averaged maxima) are calculated for the four filling levels of the lungs in the variant according to FIG. 12 during the training phase;

FIG. 14 is a graph showing another variant of the graph shown in FIG. 8, in which a singular value decomposition (SVD) applied to signal segments in order to classify the signal segments;

FIG. 15 is a view showing how the singular value decomposition is applied during the training phase in the variant according to FIG. 14;

FIG. 16 is a view showing how four shape parameter values (averaged signal segments) are calculated during the training phase in the variant according to FIG. 14;

FIG. 17 is a graph showing a possible process for calculating a reference signal segment from sum signal segments during the training phase;

FIG. 18 to FIG. 23 show a sequence in which different bands are detected after a wavelet transformation.

### DESCRIPTION OF PREFERRED EMBODIMENTS

Referring to the drawings, the process according to the present invention is used in one application to automatically control a mechanical ventilator. This ventilator assists the spontaneous breathing of a patient or replaces same completely if the patient is sedated. This work of the ventilator, especially the times and amplitudes of the ventilation strokes, shall be synchronized—if present—with the spontaneous breathing of the patient.

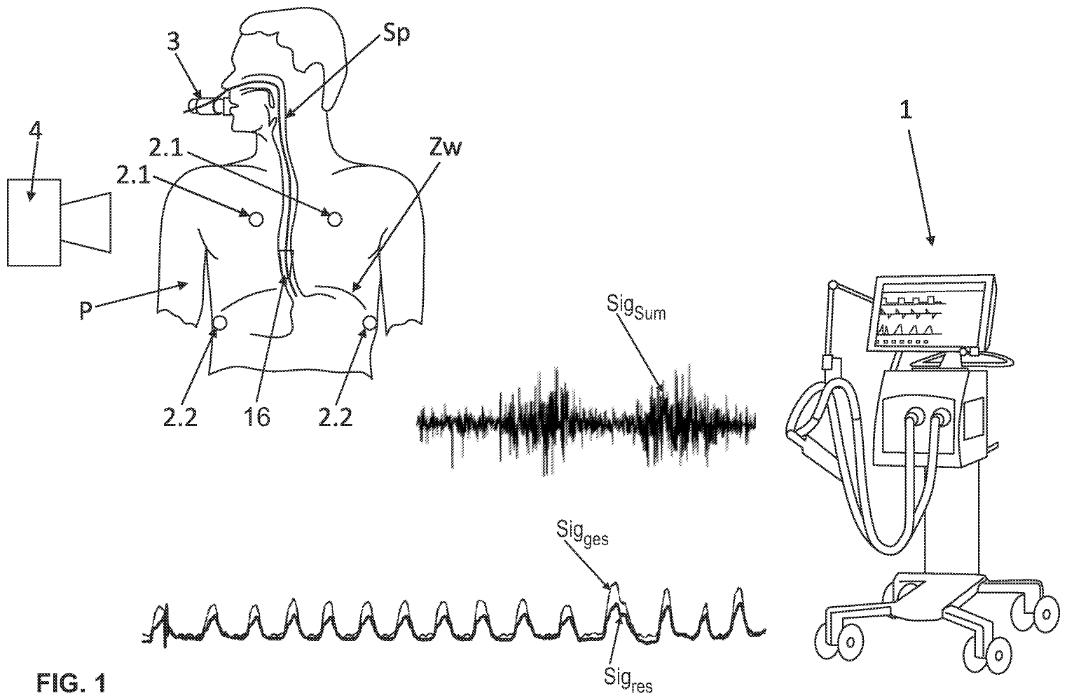

FIG. 1 shows schematically
a patient P to be ventilated,
the esophagus Sp of this patient P,
the diaphragm Zw of this patient P,
a ventilator 1 for ventilating the patient P,
a first set 2.1 of measuring electrodes, which is arranged on the chest of the patient P in a position close to the heart and at a distance from the diaphragm,
a second set 2.2 of measuring electrodes, which is arranged on the abdomen of the patient P in position at a distance from the heart and close to the diaphragm,
a pneumatic sensor 3 in front of the mouth of the patient P, which measures the flow Vol′ of gas into and out of the airway, i.e., the volume per unit of time, and optionally the airway pressure P_{aw},
optionally a pneumatic sensor 16 in the esophagus Sp of the patient P and
an optional video camera 4, which is directed from the top onto the thoracic region and/or on the abdominal region of the patient P and generates in a contactless manner measured values in the form of images sequences, from which the current lung filling level of the patient P can be determined by image processing.

A signal processing unit 5, which preferably belongs to the ventilator 1, generates a sum signal Sig_{Sum} by processing measured values of the sensors 2.1 and 2.2 and/or of the pneumatic sensor 3 and/or of the optical sensor 4. This sum signal Sig_{Sum} results from a superimposition of a respiratory signal Sigᵣₑₛ and a cardiogenic signal Sigₖₐᵣ. The respiratory signal Sigᵣₑₛ describes in this application the intrinsic breathing activity of the patient P. This respiratory signal Sigᵣₑₛ is used for controlling the ventilator 1 and is the wanted signal. The cardiogenic signal Sigₖₐᵣ is caused by the cardiac activity of the patient P and is in this application an unwanted signal. The spontaneous breathing of the patient P, which is described by the respiratory signal Sigᵣₑₛ, as well as the mechanical ventilation by the ventilator 1, generate an overall breathing and ventilation of the patient P, which is described by an overall signal Sig_{ges}.

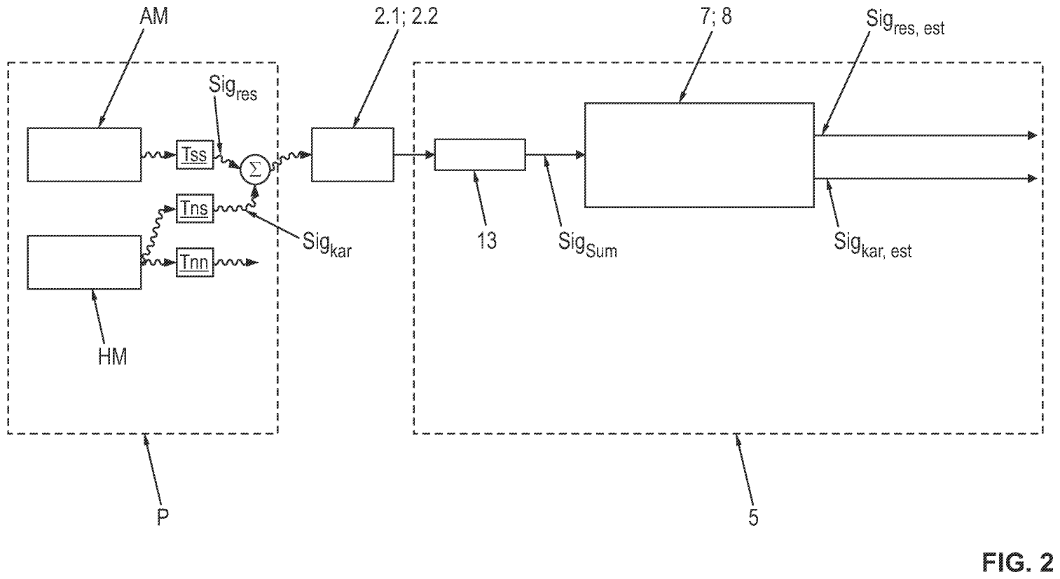

FIG. 2 shows schematically and in a simplified form how the respiratory signal Sigᵣₑₛ and the cardiogenic signal Sigₖₐᵣ are determined from the sum signal Sigₛᵤₘ. In this example the estimated (representative) cardiogenic signal Sig_{kar.est} is subtracted from the sum signal Sigₛᵤₘ, and the difference is used as an estimated (representative) respiratory signal Sig_{res,est}. Some components essential for the present invention are not shown in FIG. 2. The signal processing unit 5 yields an estimate Sig_{res,est} for the respiratory signal Sigᵣₑₛ and an estimate Sig_{kar,est} for the cardiogenic signal Sigₖₐᵣ. The estimate ideally agrees with the actual signal, i.e., ideally Sigᵣₑₛ=Sig_{res,est} and Sigₖₐᵣ=Sig_{kar,est}. Furthermore, ideally Sig_{Sum}=Sigₖₐᵣ+Sigᵣₑₛ=Sig_{kar,est}+Sig_{res,est}, i.e., ideally Sigᵣₑₛ=Sig_{Sum}+Sig_{kar,est}.

The breathing muscles AM of the patient P generate a breathing activity. The heart muscle HM generates a cardiac activity. The intrinsic breathing activity is transmitted in the body of the patient P via a transmission channel Tss to a summation point Σ, where—stated in a simplified manner—the respiratory signal Sigᵣₑₛ appears behind the transmission channel Tss. The cardiogenic signal Sigₖₐᵣ is transmitted via a transmission channel Tns to the summation point Σ, and the cardiogenic signal Sigₖₐᵣ appears behind the transmission channel Tns. The transmission channels Tss and Tns thus influence the measured breathing activity and the measured cardiac activity. The signals Sigᵣₑₛ and Sigₖₐᵣ are superimposed to one another—simply speaking—in this summation point Σ. In addition, a transmission channel Tnn is shown. The subscript s designates the wanted signal, and the subscript n (noise) the unwanted signal.

The sensors 2.1 and 2.2 generate respective electrical measured values, as a rule, electrical voltages. A signal processor 13 with an amplifier and with an analog-digital converter processes these electrical measured values. The signal processor 13 preferably carries out, in addition, a baseline filtering, especially in order to compensate electrochemical processes in the measuring electrodes 2.1 and 2.2 on the skin of the patient P and other low-frequency potential differences by calculation. These processed measured values act in the exemplary embodiment as the sum signal or a sum signal Sigₛᵤₘ. The sensors 2.1 and 2.2 are therefore sum signal sensors forming a sum signal sensor device in the sense of the present invention. The pneumatic sensor 3 and the optical sensor 4 also yield measured values, from which a sum signal is generated in variants of the present invention and another parameter value is generated in other variants.

The signal processing unit 5, which preferably forms a part of the ventilator 1, determines from this sum signal Sigₛᵤₘ the estimate Sig_{res.est} for the respiratory signal Sigᵣₑₛ being sought. The signal processing unit 5 determines for this an estimate Sig_{kar,est} for the cardiogenic signal Sigₖₐᵣ, which acts in this application as an unwanted signal. In other applications, the estimated cardiogenic signal Sig_{kar,est} is used as a wanted signal, and the respiratory signal Sigᵣₑₛ is an unwanted signal. Or else both signals Sigᵣₑₛ and Sigₖₐᵣ are wanted signals.

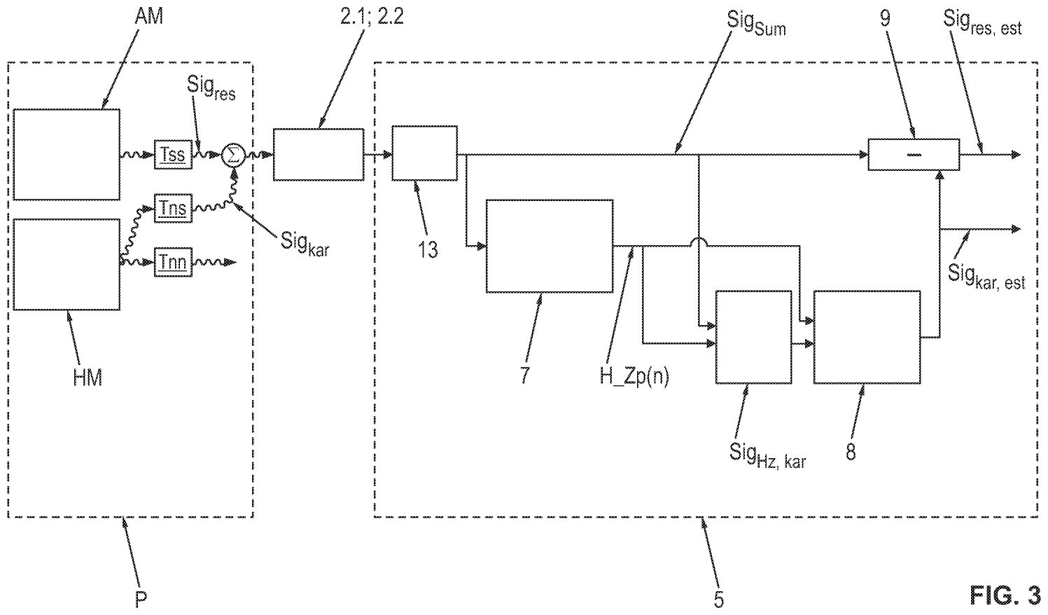

FIG. 3 shows the principle of how the influence of the cardiogenic signal Sigₖₐᵣ on the sum signal Sig_{Sum} is compensated by calculation during a use phase. Essential components of the present invention are also not shown in FIG. 3.

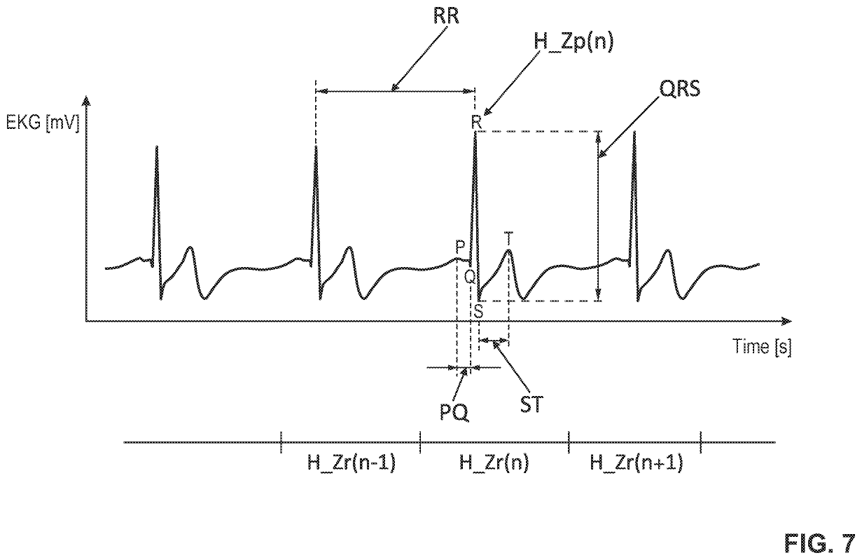

The cardiogenic signal segment Sig_{Hz,kar} describes an estimated segment of the cardiogenic signal in the course of a single heartbeat. A heartbeat time detector 7 detects the respective time H_Zp(n) of the nth detected heartbeat (n=1, 2, 3, . . . ). This heartbeat time detector 7 detects, for example, the so-called R peak or also the QRS curve in the sum signal Sig_{Sum} or also in a signal that is obtained exclusively from measured values of the set 2.1 of measuring electrodes that is placed close to the heart, cf. FIG. 7. A reconstructing unit 8 combines these estimated signal segments Sig_{Hz,kar} into a reconstructed cardiogenic signal Sig_{kar,est}, which is used as the estimate Sig_{kar,est} for the cardiogenic signal Sigₖₐᵣ, with the use of the detected heartbeat times H_Zp(x), H_Zp(x+1), . . . . This reconstructed cardiogenic signal Sig_{kar,est} is ideally equal to the actual cardiogenic signal Sigₖₐᵣ, which is generated by the heart muscles HM of the patient P. A compensating unit 9 compensates the influence of the cardiogenic signal Sigₖₐᵣ on the sum signal Sig_{Sum} by calculation. For example, the compensating unit 9 subtracts the reconstructed cardiogenic signal Sig_{kar,est} from the sum signal Sig_{Sum}. The compensating unit 9 yields in the ideal case the respiratory signal Sigᵣₑₛ being sought, i.e., ideally Sigᵣₑₛ equals Sig_{Sum}−Sig_{kar,est}.

The respiratory signal Sigᵣₑₛ and/or the cardiogenic signal Sigₖₐᵣ are influenced by at least one respective anthropological variable in the body of the patient P. A measurable parameter, which acts on at least one above-described transmission channel Tss, Tns and is therefore called transmission channel parameter, correlates with the anthropological variable or with at least one anthropological variable. This influence is not taken into consideration in FIG. 2 and FIG. 3. It will be described below how this is taken into account according to the present invention.

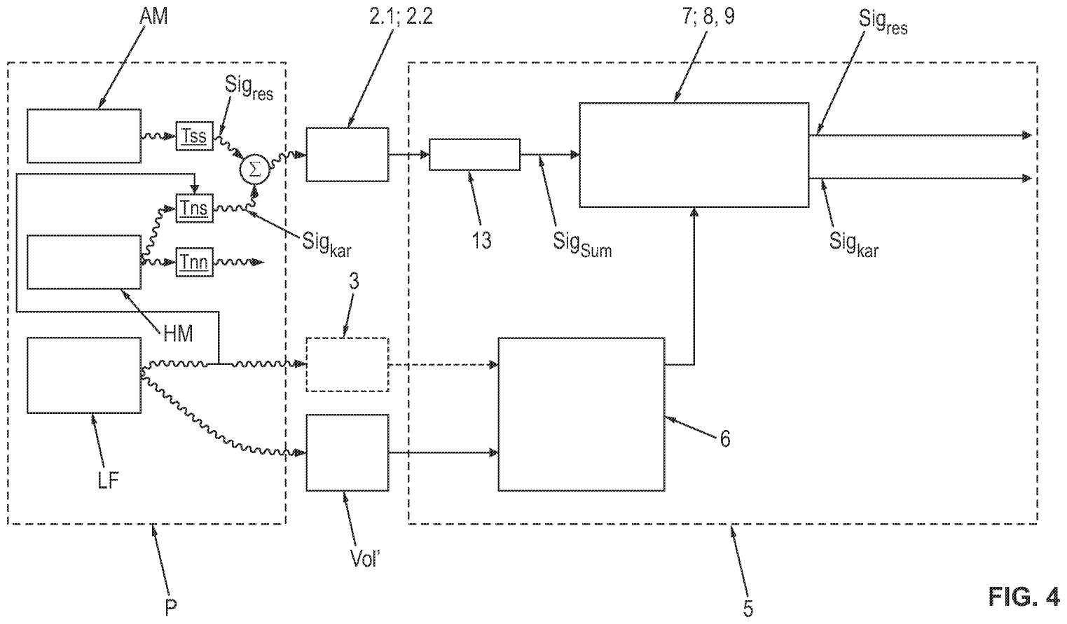

FIG. 4 shows as an example an influence on the transmission channel Tns from the breathing muscles AM, which are the signal source for the respiratory signal Sigᵣₑₛ, to the sensor 2.1, 2.2, namely, the lung filling level, LF. The current filling level LF of the lungs of the patient P changes the distance between the breathing muscles AM and the sensor 2.1, 2.2 and hence the length and also the other properties of the transmission channel Tns. The current lung filling level, LF, is correlated with the flow Vol′ of breathing air or of another gas into and out of the airway of the patient P, i.e., with the volume fed or removed per unit of time. The pneumatic sensor 3 in front of the mouth of the patient P is capable of measuring this volume flow Vol′. In the exemplary embodiment, This measured volume flow Vol′ is integrated over time and the run time of gas between the sensor 3 and the mouth as well as between the mouth and the lungs of the patient P as well as optionally the elasticity of the lungs and the resistance offered by the airway of the patient P to the flow of breathing air are additionally taken into consideration. The respective current value is determined in this manner for the transmission channel parameter LF.

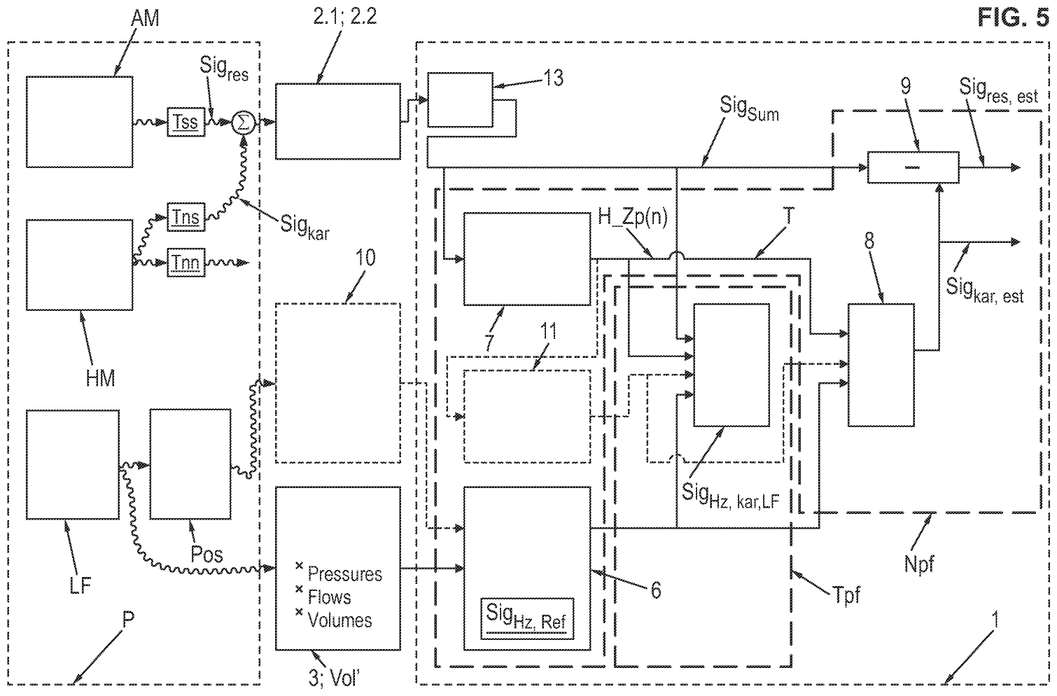

FIG. 5 shows how the principle illustrated in FIG. 4, namely, the taking into account of the lung filling level, LF, is applied to the principle illustrated in FIG. 3 in order to compensate the influence of the cardiogenic signal Sigₖₐᵣ on the sum signal Sig_{Sum}. A use path Npf and a training path Tpf are shown in FIG. 5 and in figures following it. The use path Npf describes the steps and the components used during the use phase Np, and the training path Tpf describes the steps and the components used during the training phase Tp and during the subsequent adaptation phase Ap, which overlaps with the use phase Np.

An additional transmission channel parameter, namely the position Pos of a measuring electrode 2.1 or 2.2 relative to the signal source for the cardiogenic signal Sigₖₐᵣ, is optionally taken into account in the example shown in FIG. 5. A mechanical sensor 10, for example, an acceleration sensor or a strain gauge, measures the position Pos of measuring electrodes 2.1 or 2.2 relative to a predefined reference point in or at the body of the patient P and hence relative to the heart, i.e., to the signal source HM for the cardiogenic signal Sigₖₐᵣ. Repeatedly a respective value for the transmission channel parameter LF is derived from the measured values from the sensor 3, and a value for the transmission channel parameter Pos is derived from the measured values from the sensor 10.

A third transmission channel parameter, which does not require an additional sensor, especially the length of a heartbeat or also the time period between the two characteristic times H_Zp(x), H_Zp(x+1) of two consecutive heartbeats or the time interval between two signal peaks, e.g., the P peak and the T peak, of the segment Abs.x, Abs.y, . . . of the sum signal Sig_{Sum}, which occurs in the course of a single heartbeat, is optionally taken into consideration. This time period may remain the same over time or vary over time. A heartbeat time period detector 11 analyzes the sum signal Sig_{Sum} and the detected heartbeat times H_Zp(x), H_Zp(x+1), . . . and calculates the time interval between two consecutive heartbeat times.

A heartbeat time detector 7 detects, in turn, the respective time H_Zp(n) (n=1, 2, . . . ) of each heartbeat. A signal estimating unit (a signal representation unit) 6 receives the measured values from the two sensors 3 and 10 and calculates from these the respective current value, which the transmission channel parameter LF or Pos assumes at the heartbeat time H_Zp(x).

Depending on the measured value for the lung filling level, LF, and optionally on the measured value for the relative position Pos during a heartbeat, the signal estimating unit 6 calculates during the use phase Np a respective estimated signal segment Sig_{Hz,kar.LF} of the cardiogenic signal Sigₖₐᵣ in the course of this heartbeat for each heartbeat, wherein the estimated signal segment Sig_{Hz,kar.LF} depends on the lung filling level, LF, at this heartbeat as well as optionally on the position Pos of the measuring electrode 2.1 or 2.2 and/or on the time interval RR between two heartbeats. This signal segment Sig_{Hz,kar.LF} estimated as a function of at least one transmission channel parameter, varies, as a rule, from one heartbeat to the next. The estimated signal segments Sig_{Hz,kar.LF} are combined into the estimated cardiogenic signal Sig_{kar,est} with the use of the heartbeat times.

In one embodiment, each estimated signal segment Sig_{Hz,kar.LF} has the same length. The intermediate space in the estimated signal Sig_{kar,est} is bridged over by a smoothing. In another embodiment, the respective time period H_Zr(x), H_Zr(x+1), . . . of each heartbeat is measured during the use phase Np, and the estimated signal segment Sig_{Hz,kar.LF} is adapted to this heartbeat time period by stretching or compression.

The signal estimating unit 6 has in one embodiment reading access to a predefined standard reference signal segment Sig_{Hz,Ref}, which is stored in a library. This segment describes an average segment of the cardiogenic signal Sigₖₐᵣ in the course of a single heartbeat. This standard reference signal segment Sig_{Hz} was generated, for example, in advance by measurements on different patients. It contains at least one and preferably a plurality of shape parameters, which change the geometric shape of the reference signal segment Sig_{Hz,Ref}. The influence of a transmission channel parameter is taken into account indirectly by at least one shape parameter, which will be described farther below.

Examples of shape parameters are, cf. FIG. 7:
the duration of the QRS phase,
the QRS amplitude,
the respective amplitude of the Q peak, R peak, S peak and
the time period between the P peak and the T peak.

Due to a respective shape parameter value each being introduced into the shape parameter or each shape parameter of this standard reference signal segment Sig_{Hz,Ref}, a parameterized cardiogenic estimated signal segment Sig_{Hz,kar.LF} is generated, which describes the estimated cardiac activity in the course of an individual heartbeat and depends in this example on the lung filling level, LF, and optionally on the position Pos. This parameterized standard reference signal segment Sig_{Hz,kar.LF} is shown in FIG. 5 as the expected signal segment Sig_{Hz,kar} in the course of an individual heartbeat, as this is shown in FIG. 3.

These shape parameter values depend in the example according to FIG. 5 on the current value of the lung filling level, LF, on the other hand. The current lung filling level, LF, is measured in the example according to FIG. 5 by at least one pneumatic sensor 3, and this pneumatic sensor 3 measures the volume flow Vol′ and optionally also the airway pressure P_{aw}. The shape parameter values optionally depend, in addition, on the position Pos.

In one embodiment, the signal estimating unit 6 calculates for each shape parameter of the standard reference signal segment Sig_{Hz,Ref} and for each detected heartbeat a respective shape parameter value each, which the shape parameter assumes at the heartbeat time H_Zp(x) or during the heartbeat time period H_Zr(x). Using these shape parameter values, the signal processing unit 5 generates during the use phase from the standard reference signal segment Sig_{Hz,Ref} for each heartbeat an estimated signal segment Sig_{Hz,kar.LF}, which is adapted to the current value of the lung filling level, LF, and optionally to the current position Pos and/or other transmission channel parameter, and which describes the expected or estimated cardiogenic signal Sigᵣₑₛ in the course of this heartbeat. This is carried out for each heartbeat detected during the use phase Np.

In another embodiment, the signal estimating unit 6 determines in a library 12 a stored reference signal segment Sig_{Hz,kar,LF.1} or . . . Sig_{Hz,kar,LF.4}, which segment is associated with this lung filling level, LF.1, . . . , LF.4 and optionally to this position Pos. The signal estimating unit 6 yields the estimated signal segment Sig_{Hz,kar,LF} for a heartbeat as a function of the determined reference signal segment or of each determined reference signal segment. No standard reference signal segment Sig_{Hz,ref} is needed after the end of the training phase Tp in this embodiment. The reconstructing unit 8 combines, in both embodiments during the useful segment Np, the estimated cardiogenic signal segments Sig_{Hz,kar,LF} in the course of a respective heartbeat each into an estimated cardiogenic signal Sig_{kar,est} and uses for this the heartbeat times H_Zp(x), H_Zp(x+1), . . . , which the time detector 7 has detected. According to the embodiment of the present invention, the reconstructing unit 8 combines the estimated signal segments Sig_{Hz,kar,LF} adapted to the current filling levels of the lungs, LF, into the reconstructed cardiogenic signal Sig_{kar,est}. This is preferably repeated continually as soon as a new heartbeat is detected.

A plurality of variants of the process according to the present invention will be described below, as it is illustrated by FIG. 4 and FIG. 5. The variants differ by the sensors, from the measured values of which the sum signal Sig_{Sum} is generated, by the transmission channel parameters taken into consideration and/or by the sensors in order to measure the values of these transmission channel parameters taken into account. In one variant, estimated signal segments are not combined into the cardiogenic signal Sig_{kar,est}, but into the respiratory signal Sig_{res,est}.

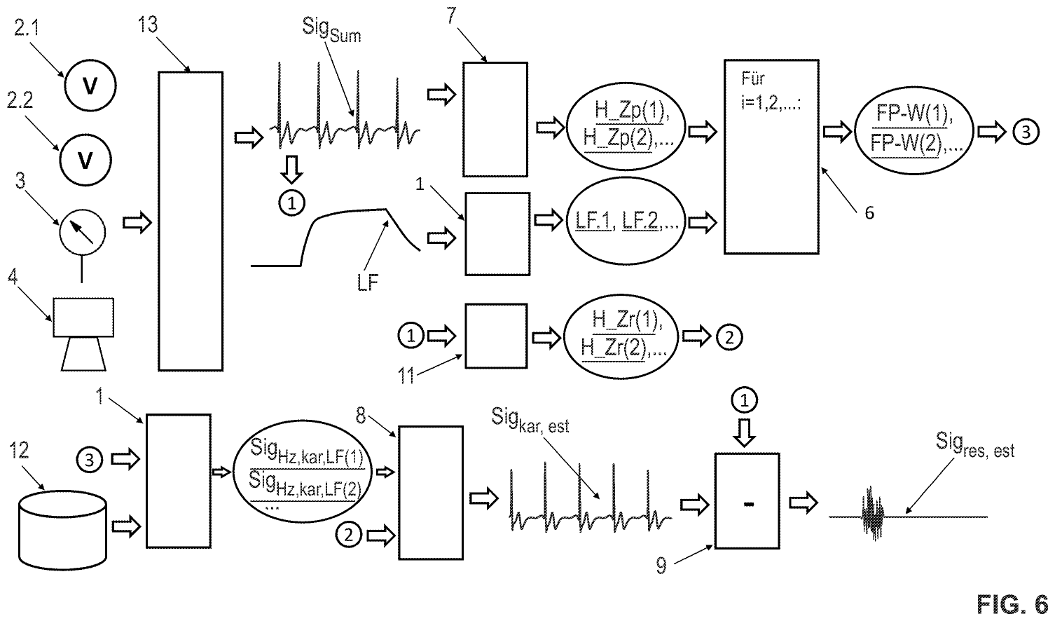

FIG. 6 shows as an example several steps that are carried out during the use phase in order to determine the estimated respiratory signal Sig_{res,est}. The following steps are shown:
The measuring electrodes 2.1 and 2.2, the pneumatic sensor 3 and/or the optical sensor 4 yield measured values.
The signal processor 13 processes the measured values from the sensors 2.1, 2.2, 3, 4 and yields a sum signal Sig_{Sum},
The heartbeat time detector 7 detects the respective heartbeat time H_Zp(n) of the nth detected heartbeat. The heartbeat time detector 7 analyzes for this the sum signal Sig_{Sum} and/or measured values from the measuring electrode set 2.1 located close to the heart.
The signal estimating unit 6 has reading access to the library 12, in which different reference signal segments Sig_{Hz,kar,LF.1}, . . . , Sig_{Hz,kar,LF.4} for different possible filling levels of the lungs, LF.1, . . . , LF.4 are stored.
The signal estimating unit 6 determines for each heartbeat from the measured heartbeat times H_Zp(x1), H_Zp(x2), . . . and from the measured filling levels of the lungs, LF.1, LF.2 a respective set of shape parameter values FP-W(1), FP-W(2), . . . and herefrom an estimated signal segment Sig_{Hz,kar.LF(x1)}, Sig_{Hz,kar,LF(x2)}, . . . each, for example by introducing the shape parameter values FP-W(1), FP-W(2) into a standard reference signal segment Sig_{Hz,Ref}.
The reconstructing unit 8 combines these estimated signal segments Sig_{Hz,kar.LF(x1)}, Sig_{Hz,kar.LF(x2)}, . . . into an estimated cardiogenic signal Sig_{kar,est}.
The heartbeat time period detector 11 optionally measures the respective heartbeat time period H_Zr(x), H_Zr(x+1) of each heartbeat.
The compensating unit 9 compensates by calculation the influence of the respiratory signal Sigᵣₑₛ on the sum signal Sig_{Sum}, for example, by subtracting the estimated cardiogenic signal Sig_{kar,est} from the sum signal Sig_{Sum} and/or the signal segment Sig_{HzmkarmLF(x1)}, Sig_{Hz,kar.LF(x2)} estimated for this heartbeat from the sum signal Sig_{Sum}.

FIG. 7 shows an exemplary segment of an electrical cardiogenic signal Sigₖₐᵣ in the course H_Zr(n) of a single heartbeat. The time is plotted on the x axis and the cardiogenic signal in mV is plotted on the y axis. The P peak, the Q peak, the R peak, the S peak and the T peak are shown. The cardiogenic signal Sigₖₐᵣ and therefore also the sum signal Sig_{Sum} have approximately the same course for each heartbeat in the range of the P peak through the T peak.

The R peak is used in one embodiment as a characteristic time H_Zp(n) of a heartbeat. In addition, the following geometric parameters are illustrated:
The R-R interval RR between the R peaks of two consecutive heartbeats,
the QRS amplitude QRS, i.e., the distance between the highest value and the lowest value during the time period between the Q peak and the S peak,
the P-Q time interval PQ, i.e., the time period between the P peak and the Q peak, and
the S-T time interval ST, i.e., the time interval between the S peak and the T peak.

The R-R interval RR correlates with the pulse of the patient P.

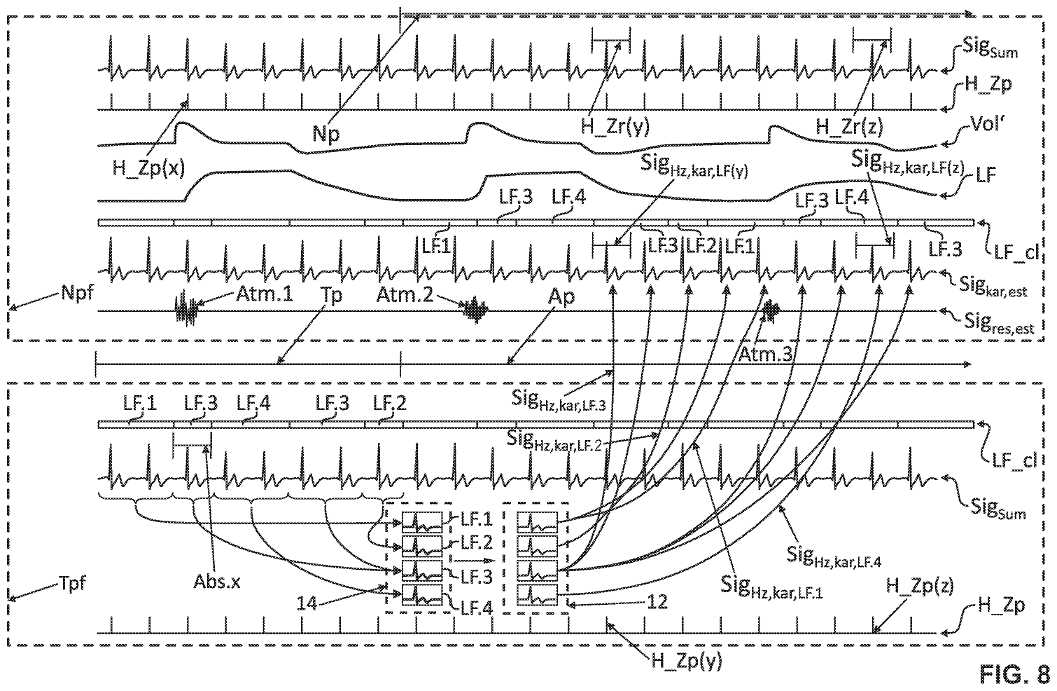

FIG. 8 shows as an example how the sample elements are generated and used according to a first variant. Shown are
The training phase Tp, during which a sample 14, optionally a library 12 and then an initial signal estimating unit 6 are generated,
the subsequent adaptation phase Ap, during which this signal estimating unit 6 is continually adapted to the sample elements obtained so far during the use phase Np, as well as
the use phase Np, during which the signal estimating unit 6 is used.

The adaptation phase Ap overlaps the use phase Np. The time is plotted from left to right on the respective x axis of each signal. The time curves of the following signals are shown:
The sum signal Sig_{Sum},
the respective characteristic heartbeat time H_Zp,
the curve of the volume flow Vol′ and
the curve of the lung filling level, LF.

In this variant, the sum signal Sig_{Sum} is generated by analyzing electrical measured values of the measuring electrodes 2.1 and 2.2. The volume flow Vol′ is measured, for example, by means of the pneumatic sensor 3, and the current lung filling level, LF, is derived from the respective volume flow Vol′ at a plurality of times. Four classes of filling levels of the lungs, namely, LF.1 (lungs almost empty, lung filling level below a first limit), LF.4 (lungs almost full, lung filling level above a second limit) and two filling levels of the lungs in between, LF.2 and LF.3, are distinguished in the example shown. A different number of classes of filling levels of the lungs, LF, and of other transmission channel parameters may, of course, be distinguished as well. The signal with the time curve that indicates the class to which the current lung filling level, LF, belongs, is designated by LF_cl in FIG. 8.

In an embodiment of the example according to FIG. 8, each sample element comprises a segment of the sum signal Sig_{Sum} in the course of an individual heartbeat, for example, segment Abs.x in the course of the heartbeat with the characteristic heartbeat time H_Zp(x). In addition, each sample element comprises a class each of the lung filling level, LF, for example, class LF.3 for the heartbeat time H_Zp(x). It is illustrated by means of a plurality of arrows in the bottom part of FIG. 8 how four classes LF.1 through LF.4 of sample elements are generated. The corresponding segments of the sum signal Sig_{Sum}, which belong to the sample elements of one class, are brought to the same length by projecting segments being cut off by calculation, and then being superimposed with the correct time. The arithmetic mean of the segments superimposed with the correct time is formed or these segments are combined in another manner into a reference signal segment, which reference signal segment is assigned to the class of filling levels of the lungs. A computer-accessible library 12 with—in this case four—stored reference signal segments Sig_{Hz,kar,LF.1}, . . . , Sig_{Hz,kar,LF.4} of the cardiogenic signal is generated hereby in the course of a heartbeat. Each reference signal segment SigSig_{Hz,kar,LF.1}, . . . , Sig_{Hz,kar,LF.4} is assigned in the library 12 to a possible lung filling level class LF.1, . . . , LF.4.

The class to which the lung filling level, LF(t), belongs at the time t=H_Zp (n) is determined during the use phase Np in one embodiment at the characteristic heartbeat time H_Zp(n) of the nth heartbeat. In one embodiment, the respective reference signal segment which is assigned to this class in the library 12, is used as the estimated signal segment Sig_{Hz,kar,LF(n)}. It describes the segment of the cardiogenic signal in the course of this heartbeat. For example, the reference signal segment Sig_{liz,kar,LF.3} for the lung filling level LF.3 is selected for the time H_Zp(y) and is used as an estimated signal segment Sig_{Hz,kar,LF(y)}, and the reference signal segment Sig_{Hz,kar,LF.4} for the lung filling level LF.4 is used for the time H_Zp(z) as the estimated signal segment Sig_{Hz,kar,LF(z).}

In another embodiment, the signal processing unit 5 calculates for each class of lung filling levels, in addition to the reference signal segment, a respective reference parameter value each, for example, as a weighted mean value or as a center or median of the transmission channel parameter values (here: lung filling levels) of this class. For example, the relative frequencies of transmission channel parameter values are used as weighting factors. The signal processing unit 5 determines during the use phase Np for each heartbeat the two reference parameter values that are closest to the transmission channel parameter value of this heartbeat and calculates the estimated signal segment for this heartbeat by smoothing, for example, an interpolation or regression.

The signal estimating unit 6 consequently yields for each heartbeat time H_Zp(y) an estimated signal segment Sig_{Hz,kar,LF(y)}, which depends on the four possible reference signal segments Sig_{Hz,kar,LF.1}, . . . Sig_{Hz,kar,LF.4}. In one embodiment, each estimated signal segment Sig_{Hz,kar,LF(y)} of the cardiogenic signal is equal to a reference signal segment Sig_{Hz,kar,LF.1}, . . . , Sig_{Hz,kar,LF.4} in the library 12. The estimated signal segment provided depends on which of the four classes LF.1, . . . , LF.4 the lung filling level LF belongs to during this heartbeat.

This is carried out after the training phase Tp, i.e., when the signal estimating unit 6 is generated. A respective standard reference signal segment predefined in advance for each detected heartbeat is preferably used for each heartbeat time before the end of the training phase Tp.

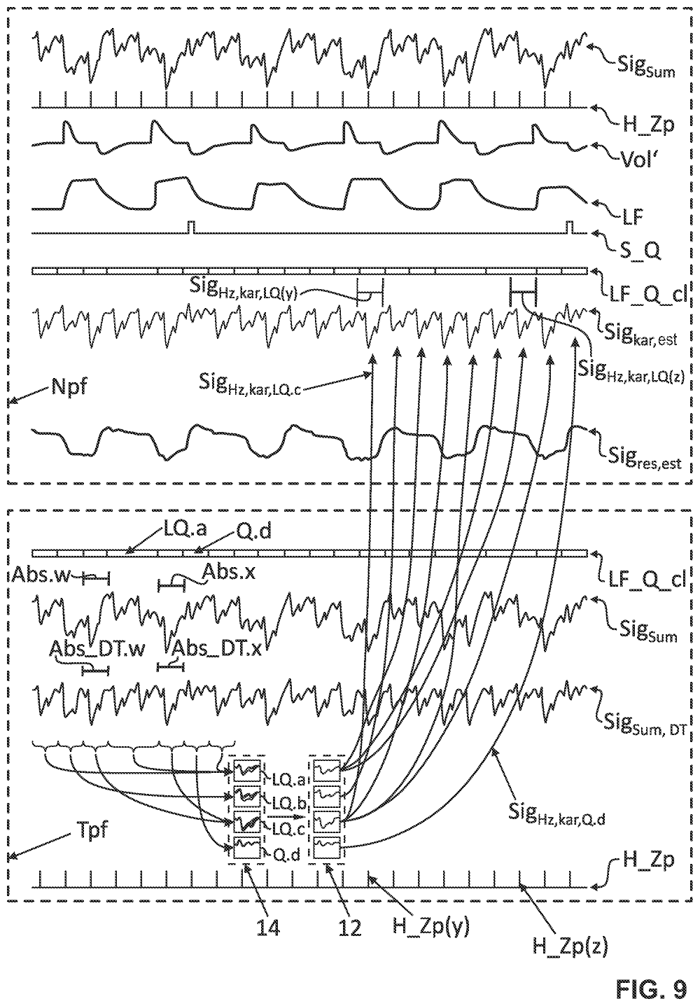

These estimated signal segments Sig_{Hz,kar,LF} are combined by the reconstructing unit 8 into the estimated cardiogenic signal Sig_{kar,est}. This estimated cardiogenic signal Sig_{kar,est} as well as the estimated respiratory signal Sig_{res,est} are shown below the curve LF_cl in FIG. 8. The estimated respiratory signal Sig_{res,est} is generated by the compensating unit 9 subtracting from the measured sum signal Sig_{Sum} the estimated cardiogenic signal Sig_{kar,est} generated by combination, i.e., Sig_{res,est}=Sig_{Sum}−Sig_{kar,est}. As can be seen, the estimated respiratory signal Sig_{res,est} usually assumes the zero value because the heart rate is several times higher than the respiration rate and the cardiogenic signal Sigkₐᵣ is several times stronger in the P-T segment of a heartbeat than the respiratory signal Sigᵣₑₛ. Three breathing processes of the patient P lead to three oscillations Atm.1, Atm.2, Atm.3 of the estimated respiratory signal Sig_{res,est} shown. FIG. 9 shows a variant of the approach shown in FIG. 8. The coordination between the spontaneous breathing and the heartbeat of the patient P, more precisely the event of whether the exhalation begins shortly before the Q wave of the next heartbeat or not, is used as an additional transmission channel parameter. The signal S_Q shows the time curve of this additional transmission channel parameter. The classes are formed depending on two transmission channel parameters, namely, the lung filling level LF and the exhalation time close to Q (yes/no).

In a possible embodiment, this leads with four classes LF.1, . . . , LF.4 for the lung filling level LF and for two classes for the exhalation time (yes and no, i.e., breathing begins and breathing does not begin shortly before the Q wave) to a total of 2×4=8 different classes. By contrast, only four classes are used in the embodiment shown. The possible values for the lung filling level LF are grouped into three classes LF.a, LF.b, LF.c. In connection with the event that the exhalation time is not close to Q, this leads to three classes LQ.a, LQ.b, LQ.c. In addition, a fourth class Q.d is introduced, namely, that the exhalation time is close to Q, regardless of how the lung filling level LF is. Furthermore, the time curve of the belonging to one of these four classes LF.a, LF.b, LF.c, Q.d is shown in FIG. 9, which is designated by LF_Q_cl.

The sum signal Sig_{Sum} is a pressure signal in this variant, which is measured in or in front of the esophagus Sp (esophagus) of the patient P, for example, with a probe or with a balloon in the esophagus Sp. The pressure signal could also be the pressure P_{aw} at the transition from a hose of the ventilator 1 to the mouth of the patient P, which is measured by the sensor 3. This pneumatic sum signal Sig_{Sum} results from a superimposition of the pneumatic respiratory signal Sigᵣₑₛ brought about by the breathing activity to a pneumatic cardiogenic signal Sigₖₐᵣ brought about by the cardiac activity.

In the variant shown, the signal processing unit 5 therefore additionally carries out a detrending in the training phase Tp and therefore in the training path Tpf. As a result, the risk that different trends would distort the combination of the sum signal segments arranged in correct time into a reference signal segment is reduced. Both the sum signal Sig_{Sum} and the processed sum signal Sig_{Sum,DT} generated by the detrending are shown in FIG. 9.

An embodiment of generating the detrending is the following: The signal processing unit determines for each heartbeat the sum signal segment Abs.w, Abs.x belonging to this heartbeat. It calculates a fitted curve, especially a fitted curve through this sum signal segment Abs.w, Abs.x. This fitted curve is generated, for example, by interpolation or as a straight line from the chronologically first to the chronologically last signal value of the sum signal segment Abs.w, Abs.x. The respective fitted curve is subtracted for each heartbeat from the sum signal segment Abs.w, Abs.x. The remaining residue, i.e., the difference, forms the processed sum signal segment Abs_DT.w, Abs_DT.x generated by the detrending. Each sample element comprises such a processed sum signal segment. These segments yield the estimated signal segments Sig_{Hz,kar,LD(y)}, Sig_{Hz,kar,LF(z)} which are combined into the processed sum signal Sig_{Sum,DT}.

The signal estimating unit 6 yields during the use phase a respective processed sum signal segment Abs_DT.w, Abs_DT.x for each detected heartbeat.

The signal estimating unit 6 yields a respective estimated signal segment Sig_{Hz,kar,LQ}, which is selected among four possible reference segments SigSig_{Hz,kar,LQ.a}, . . . , SigSig_{Hz,kar,Q.d} of the cardiogenic signal Sigₖₐᵣ, during the use phase Np for each heartbeat in one embodiment in the variant according to FIG. 9 as well, and the particular estimated signal segment which the signal estimating unit 6 provides for a heartbeat depends on the lung filling level LF and on the exhalation time during the heartbeat.

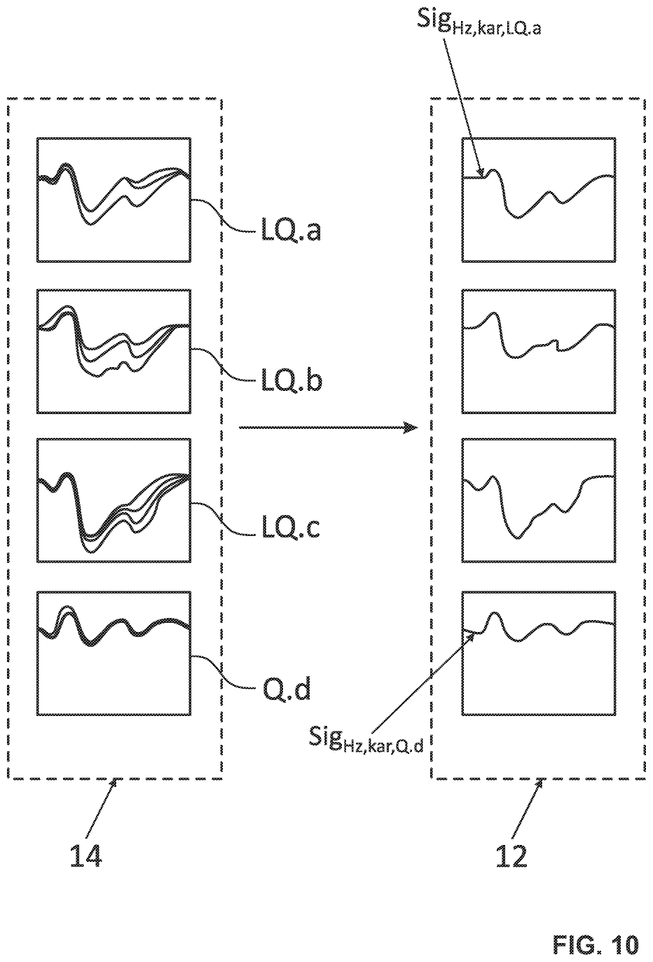

FIG. 10 shows how the four reference signal segments Sig_{Hz,kar,LQ.a}, . . . , Sig_{Hz,kar,LQ.d} of the cardiogenic signal Sigₖₐᵣ are formed for the four different classes (lung filling levels and Q values) LQ.a, LQ.b, LQ.c, Q.d. The segments of the sum signal Sig_{Sum}, which are superimposed with correct time, and which belong to the same class, i.e., here to the same lung filling level/Q value LQ.a, LQ.b., LQ.c, Q.d here, are shown in the left column of FIG. 10. The corresponding reference signal segment Sig_{Hz,kar,LQ.a}, . . . , Sig_{Hz,kar,Q.d} of the cardiogenic signal is shown in the right column for a class LF.1, . . . , LF.4, which is formed by calculating the arithmetic mean from the signal segments superimposed with correct time for a respective heartbeat. The content of the right column is stored in the library 12.

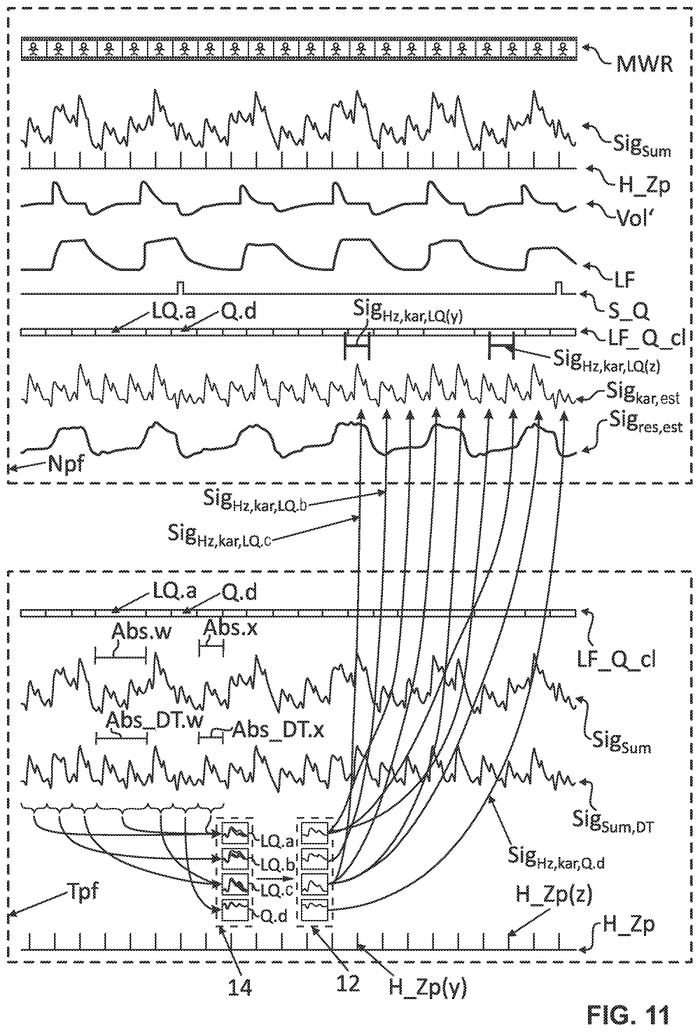

In the variant according to FIG. 11, the sum signal Sig_{Sum} is determined by an automatic image analysis of image sequences, wherein the video camera 4 is directed towards the thoracic region of the patient P and yields these image sequences. The sum signal Sig_{Sum}, which is shown in the second row of FIG. 11, is formed from a superimposition of a respiratory signal to a cardiogenic signal in this variant as well. The current lung filling level LF of the patient P is likewise derived from measured values of the pneumatic sensor 3. It is possible to use additionally signals from the video camera 4 to determine the current lung filling level, since these signals show the thoracic region of the patient P, and this region rises and falls depending on the breathing. The topmost row of FIG. 11 shows as a measured value series MWR a sequence of images that have been recorded by the video camera 4. The above-described detrending is applied to the sum signal segments in this variant as well.

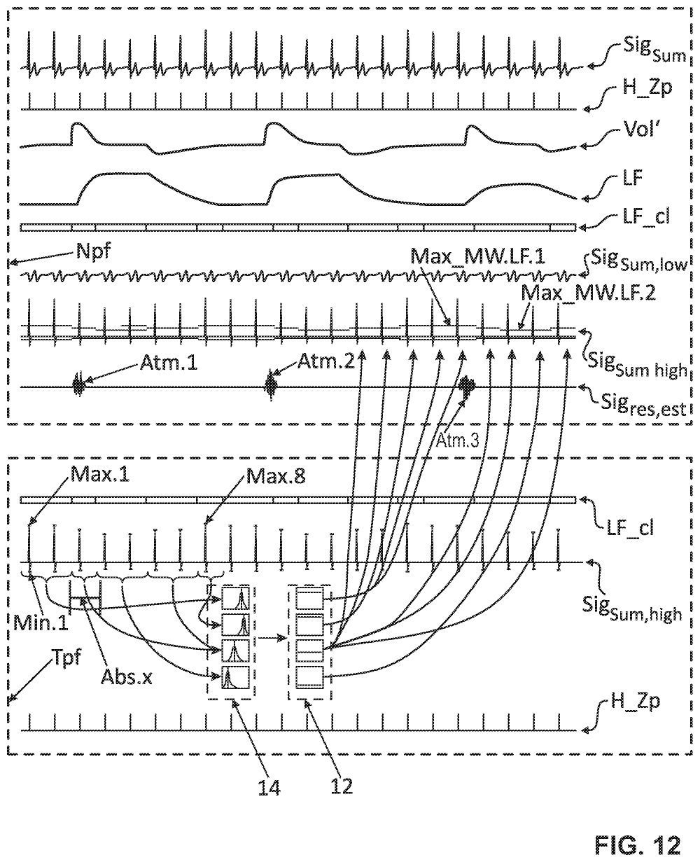

In the variant according to FIG. 12, the sum signal Sig_{Sum} is likewise generated from electrical measured values of the measuring electrodes 2.1 and 2.2. The pneumatic sensor 3 likewise measures the volume flow Vol′, and the signal processing unit 5 calculates the current lung filling level LF from a plurality of values for the volume flow Vol′. Four possible lung filling levels LF.1, . . . , LF.4 are distinguished again. No estimated cardiogenic signal Sig_{kar,est} is calculated in this variant. The estimated respiratory signal Sig_{res,est} is rather extracted by calculation from the sum signal Sig_{Sum} in another manner. No reference signal segments are used in this variant. At least two frequency ranges are predefined: A lower-frequency range and a higher-frequency range in the variant shown. For example, a frequency range results from frequencies in which an electrically measured respiratory signal (EMG) can occur, and another frequency range from frequencies, in which an electrically measured cardiogenic signal (ECG) can occur.

The sum signal Sig_{Sum} is broken down in the example shown into a respective signal component per predefined frequency range in both the training phase Tp and the use phase Np. For example, a wavelet transformation or a band filter or a low-pass filter or a high-pass filter is employed. FIG. 12 shows the signal component Sig_{Sum,low} for the lower frequency range and the signal component Sig_{Sum,high} for the higher frequency range. The signal component Sig_{Sum,low} for the lower frequency range is caused essentially, i.e., aside from a negligibly small residue, by the cardiac activity HM of the patient P and is not used for the calculation of the estimated respiratory signal Sig_{res,est}. The signal component Sig_{Sum,high} for the higher frequency range results from a superimposition of the respiratory signal Sigᵣₑₛ to a higher-frequency component of the cardiogenic signal Sigₖₐᵣ.

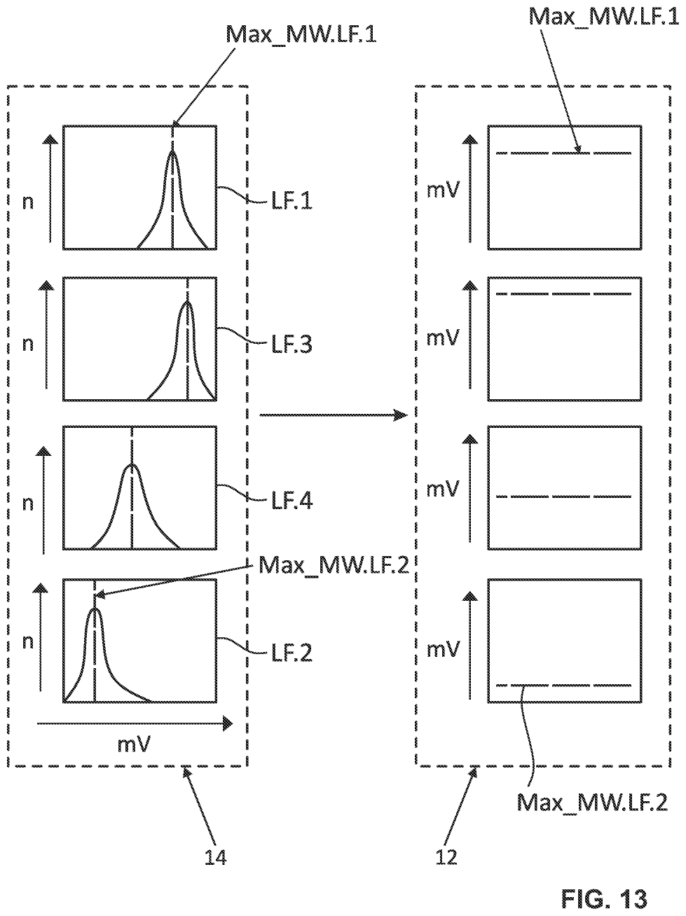

The respective maximum and the respective minimum are detected in the course of a heartbeat in the signal component Sig_{Sum,high} during the training phase Tp. For example, two maxima Max.1 and Max.8 are shown. The same is carried out for the minima. For example, a minimum Min.1 is shown. These maxima are divided into four classes of maxima in the respective heartbeat depending on the particular lung filling level LF.1, . . . , LF.4. FIG. 13 shows in the left column (sample 14) by means of four histograms the maxima of these four classes. Each rectangle corresponds to a class. The value of the maximum, i.e., a value in mV, the frequency of this maximum in a class of lung filling levels LF.1, . . . , LF.4, is plotted on the x axis of a histogram. A characteristic value, for example, an arithmetic mean or a median or maxima, is calculated for each class of maxima. Especially the two mean values or medians Max_MW.LF.1 and Max_MW.LF.2 for the two classes, which belong to the lung filling levels LF.1 and LF.2, respectively, are shown in FIG. 13. It is shown in the right column in FIG. 13 (library 12) how a respective averaged maximum, i.e., an arithmetic mean or median or maxima, is assigned as a shape parameter value to each class LF.1, . . . , LF.4 of lung filling levels. These are stored in the library 12. Furthermore, an averaged minimum, which was determined in a corresponding manner, is associated with each class. The two shape parameter values are used to parameterize a change rule (calculation rule), which will be described below.

The signal estimating unit 6 determines the sum signal segment Abs.x, the higher-frequency signal component segment and the respective lung filling level for each heartbeat in the use phase. The signal estimating unit 6 determines a respective averaged maximum as well as a respective averaged minimum, for which the signal estimating unit 6 uses the measured lung filling level LF at this heartbeat as well as the maxima and minima determined in the library 12. The signal estimating unit 6 cuts off by calculation the components that are above the averaged maxima or below the averaged minima in the segment of the higher-frequency signal component Sig_{Sum,high} that belongs to this heartbeat. These components originate with certainty essentially from the cardiogenic signal Sigₖₐᵣ and contain no respiratory components that is to be taken into account. The cutting off is illustrated in FIG. 12 on the basis of the two averaged maxima Max_MW.LF.1 and Max_MW.LF.2 stored in the library 12. The remaining components, i.e., the components of the higher-frequency signal component Sig_{Sum,high} that are located between the weighted minimum and the weighted maximum, originate from the respiratory signal Sigᵣₑₛ and are preferably smoothed by calculation. The gaps formed due to the cutting off are set, for example, at zero, or they are interpolated in a suitable manner between the remaining components. A respective signal component Sig_{Hz,res,LF(y)}, Sig_{Hz,res,LF(z)}, . . . , which describes the estimated respiratory signal in the course of this heartbeat, is generated in this manner for each heartbeat. The reconstructing unit 8 combines these signal segments Sig_{Hz,res,LF(y)}, Sig_{Hz,res,LF(z)}, lung filling level into the estimated respiratory signal Sig_{res,est}.

Averaged maxima and averaged minima are used in this example as shape parameter values of a class of transmission channel parameter values (here: lung filling level end LF.1, . . . , LF.4). These shape parameter values are used in this variant to parameterize a predefined change rule. The parameterized change rule changes a respective segment Abs.x, Abs.y of the sum signal Sig_{Sum}—in this variant: a segment of the higher-frequency signal component Sig_{Sum,high}. The change comprises in this variant the step of cutting off signal components above the maxima and below the minima.

It is also possible to use additional or other arithmetic shape parameters and hence other change rules, e.g., averaged first and/or second derivatives. It is also possible to use weighting factors and/or a “soft threshold.” A segment of the sum signal Sig_{Sum}, which segment belongs to a heartbeat, or of a signal component in the segments in which the slope of the sum signal Sig_{Sum} is below a predefined limit, is stretched in another embodiment. Due to the variant shown in FIG. 12 and FIG. 13, an estimated respiratory signal Sig_{res,est}, for which a higher-frequency signal component Sig_{Sum,high} is used, is calculated. The described process can also be applied to calculate an estimated cardiogenic signal Sig_{kar,est}. The process is applied correspondingly to the lower-frequency signal component Sig_{Sum,low} for this application. A respective estimated signal segment Sig_{Hz,kar.LF} of the cardiogenic signal Sig_{kar,est} is preferably calculated for each heartbeat. The segment of the lower-frequency signal component Sig_{Sum,low}, which segment belongs to this heartbeat, as well as the areas of the higher-frequency signal component Sig_{Sum,high} that are above the averaged maximum or below the averaged minimum for this heartbeat are combined for this purpose into the signal segment Sig_{Hz,kar,LF} for a heartbeat. The reconstructing unit 8 combines these estimated signal segments Sig_{Hz,kar,LF} into the estimated respiratory signal Sig_{kar,est}.

In a preferred application of the variant that is shown by FIG. 12 and FIG. 13, two frequency ranges are predefined, namely, a frequency range from f1 to f2 for the ECG signal (cardiogenic signal) and a frequency range from f3 to f4 for the EMG signal (respiratory signal). Now: f1<f3<f2<f4, i.e., the two frequency ranges overlap in the range from f3 to f2. The sum signal Sig_{Sum} is divided by calculation into three signal components, namely, a signal component for the frequency range from f1 to f3, a signal component for the overlapping frequency range from f3 to f2, and a signal component for the frequency range from f2 to f4. The lower-frequency signal component in the range from f1 to f3 is essentially a cardiogenic signal, i.e., the respiratory component in the lower-frequency signal component may be ignored. The high-frequency signal component in the range from f2 to f4 is essentially a respiratory signal, and the medium-frequency signal component in the range from f3 to f2 results from a superimposition of the respiratory signal to the cardiogenic signal, which superimposition is to be taken into account. The process just described is carried out only for this overlapping frequency range from f3 to e, i.e., especially the two signal components Sig_{Sum,high} and Sig_{Sum,low} are formed. The estimated respiratory signal Sig_{res,est} is combined from the component in the high-frequency range from f2 to f4 as well as from the respiratory signal obtained as just described in the overlapping frequency range from f3 to f2. The estimated cardiogenic signal Sig_{kar,est} is correspondingly combined from the component in the lower-frequency range from f1 to f3 as well as from the cardiogenic signal obtained as just described in the overlapping frequency range from f3 to f2.

In the embodiments just described, the signal processing unit 5 receives a plurality of measured values from at least one sensor, wherein this sensor is not a sum signal sensor 1, 2.1, 2.2, 3, 4, and it generates by signal processing the transmission channel parameter value or each transmission channel parameter value from these measured values. It is also possible that the signal processing unit 5 calculates the value of at least one transmission channel parameter and measures it by the calculation by the signal processing unit 5 analyzing the sum signal Sig_{Sum}. Another sensor for the transmission channel parameter is thus unnecessary for this transmission channel parameter.

Possible transmission channel parameters, which can be measured by calculation and without a separate physical sensor, are shown in FIG. 7, namely,
the R-R interval RR,
the QRS amplitude QRS,
the P-Q time interval PQ,
the P-T time interval and
the S-T time interval ST.

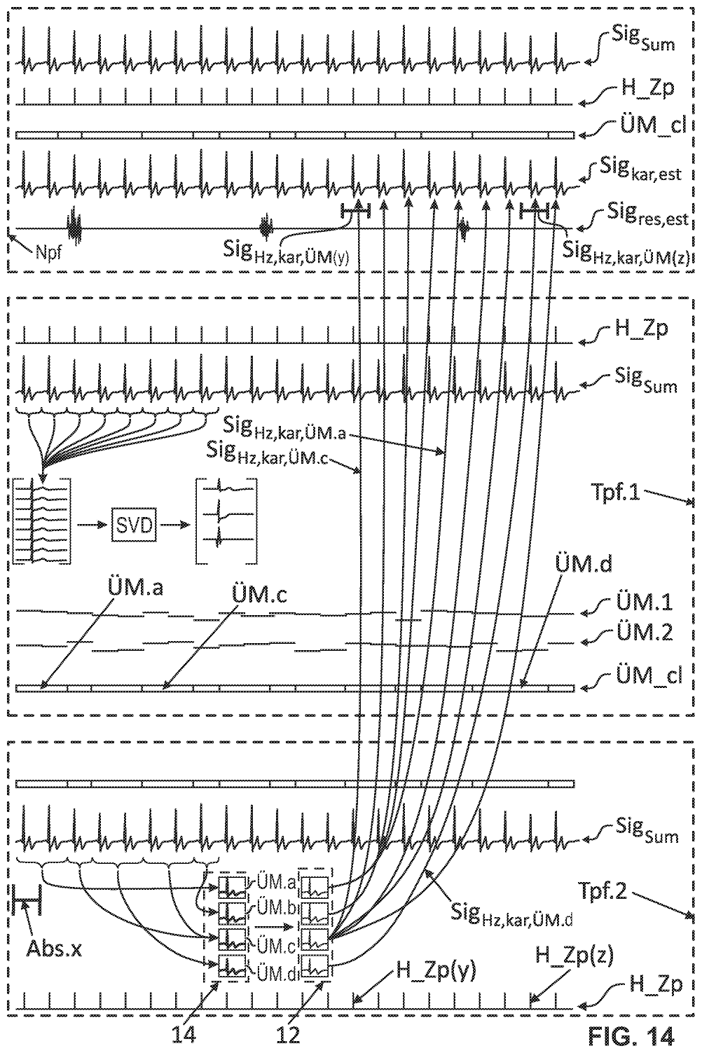

FIG. 14 through FIG. 16 show another variant, in which no additional physical sensor is needed to measure a transmission channel parameter. The basic idea of this variant is that at least one reference curve, preferably two or three reference curves, are determined before the beginning of the training phase Tp or else during the training phase Tp. The signal processing unit 5 calculates in the use phase Np a respective individual agreement value, i.e., a value for the agreement between the sum signal segment and the reference curve, for each sum signal segment Abs.x, Abs.y, . . . and each reference curve. Each sum signal segment Abs.x, Abs.y, . . . is preferably standardized in advance. The signal processing unit 5 calculates from the individual agreement values an overall agreement value. This overall agreement value acts in this variant as the transmission channel parameter or a transmission channel parameter. As in the variants described above, the signal processing unit 5 has reading access to a library 12, in which a respective reference signal segment is stored for each class of transmission channel parameter values, in this additional variant as well. In this case, each class is a range of possible overall agreement values. Depending on the calculated overall agreement values between a sum signal segment for a heartbeat and the reference curves V.1, V.2, . . . used, the signal processing unit 5 selects in the use phase Np for each heartbeat at least one respective reference signal segment from the library 12 and uses it as the estimated signal segment Sig_{Hz,kar,ÜM} for this heartbeat or yields an estimated signal segment Sig_{Hz,kar,ÜM} depending on the selected reference signal segments. The signal processing unit 5 combines the estimated signal segments Sig_{Hz,kar,ÜM} provided in this manner with the use of the heartbeat times into the estimated cardiogenic signal Sig_{kar,est} or compensates the influence of the cardiac activity on the sum signal and uses the provided estimated signal segments and the heartbeat times for the compensation.

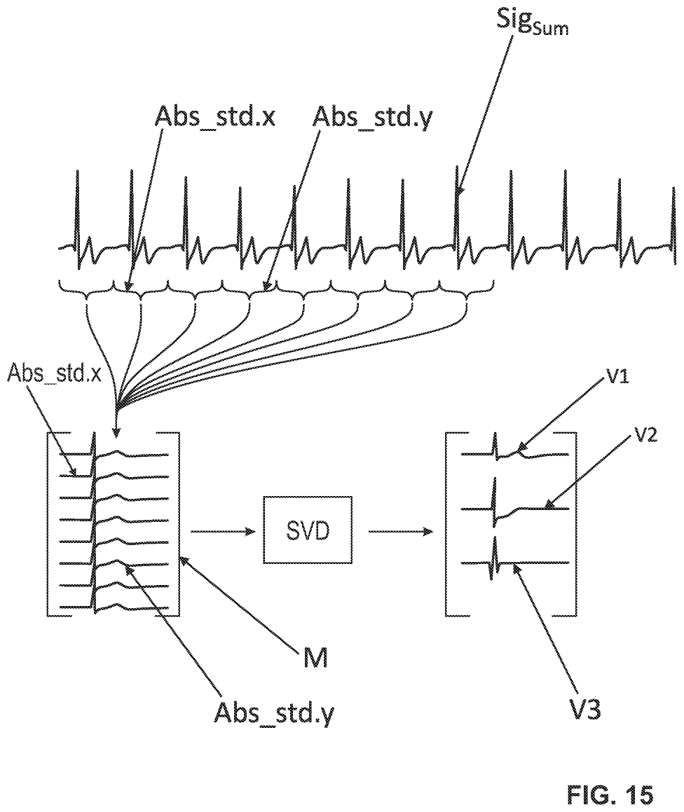

An embodiment of this variant will be explained below with reference to FIG. 14 through FIG. 16. The sum signal Sig_{Sum} is likewise divided into sum signal segments Abs.x, Abs.y, . . . , namely, into one signal segment for each heartbeat. These sum signal segments may have different lengths. By the signal processing unit cutting off parts of the sum signal segments when needed, it generates a sample, in which the sample elements comprise segments of equal length of the sum signal Sig_{Sum}. The relative times of the five peaks (P peak through T peak, see FIG. 7) of these signal segments differ from one another as little as possible. These equal-length signal segments, arranged with the correct time, will hereinafter be called standardized signal segments and are designated by Abs_std.x, Abs_std.y, . . . in FIG. 15.

These standardized signal segments Abs_std.x, Abs_std.y, . . . are arranged in a matrix M. Each row of this matrix represents a heartbeat and each column a scanning time. The signal processing unit applies to the set of these standardized signal segments in a first part Tpf.1 of the training path Tpf a singular value decomposition (SVD) or also a principal component analysis (PCA). This step yields a plurality of reference curves in a decreasing order, wherein the order depends decreasingly on an agreement value. The first reference curve V.1 agrees with the standardized signal segments most strongly, etc. The three most important reference curves V.1 through V.3 are shown in FIG. 15 in a decreasing order from top to bottom. The standardized signal segments can be reconstructed again from these reference curves.

The reference curves V.1, V.2 are predefined in an alternative embodiment.

The signal processing unit 5 classifies next the standardized sum signal segments Abs_std.x, Abs_std.y, i.e., in a second part Tpf.2 of the training path Tpf. Only the two most important reference curves V.1 and V.2 are used for this in the example being shown. It is also possible to use more than two reference curves. The signal processing unit 5 calculates for each sum signal segment Abs_std.x, Abs_std.y, . . . a respective value each for the agreement between this standardized sum signal segment and the reference curve V.1, V.2 used. For example, it calculates the scalar product between the standardized sum signal segment Abs_std.x, Abs_std.y, . . . and the reference curve V.1, V.2. The time course ÜM.1 of the individual agreement value for the first reference curve V.1 and the time course ÜM.2 of the individual agreement value for the second reference curve V.2 are shown in FIG. 14. The signal processing unit 5 then classifies each standardized sum signal segment on the basis of the two calculated individual agreement values. Two classes each of individual agreement values are used in the example shown per reference curve V.1, V.2, so that the standardized reference curves are groups into a total of 2*2=4 groups. These are called ÜM.a, . . . , ÜM.d. Furthermore, the time curve ÜM_cl of this classification is shown in FIG. 14.

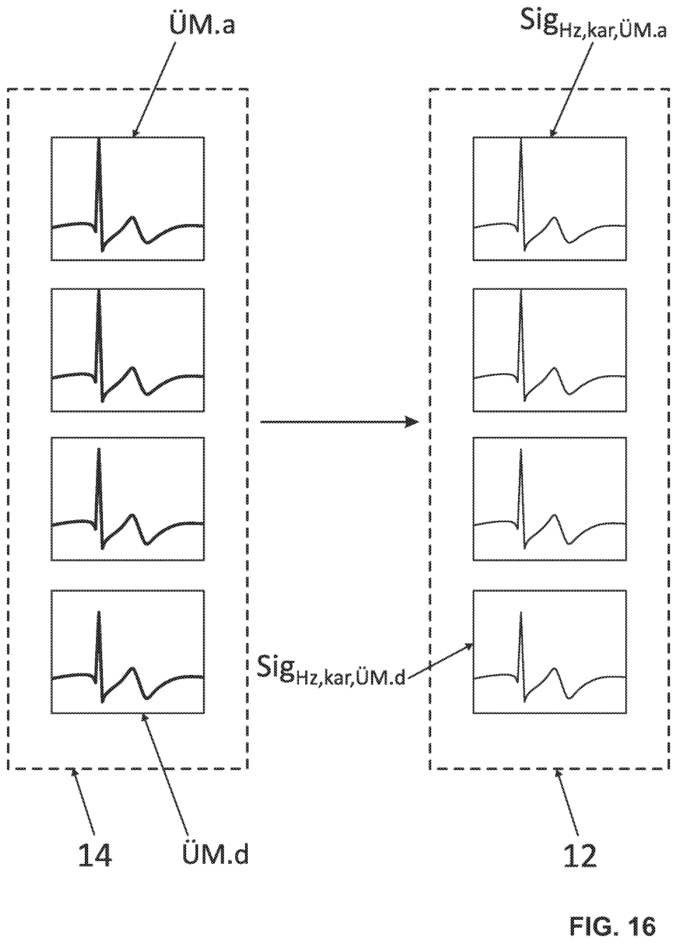

FIG. 16 shows in the left column (sample 14) the standardized sum signal segments Abs_std.x, Abs_std.y, . . . , which are divided into the four classes ÜM.a, ÜM.dThe signal processing unit 6 aggregates the standardized signal segments Abs std.x, Abs std.ye, . . . of a class ÜM.a, . . . , ÜM.d into a respective reference signal segment Sig_{Hz,kar,ÜM.a}, . . . , Sig_{Hz,kar,ÜM.d} each per class, for example, by forming for each relative scanning time the mean value or the median over the standardized signal segments Abs_std.x, Abs_std.y of this class. The library 12 with four reference signal segments Sig_{Hz,kar,ÜM.a}, . . . , Sig_{Hz,kar,ÜM.d} in this case is shown in the right column. The signal processing unit 5 generates in the use phase Np a standardized sum signal segment for each detected heartbeat from the corresponding sum signal segment Abs.x, Abs.y, . . . and calculates the respective individual agreement value between this standardized sum signal segment and each reference curve V.1, V.2, . . . , for example, as a scalar product. These two (or three) individual agreement values are summed up by the signal processing unit 5 into a preferably two-dimensional overall agreement value. Depending on this overall agreement value ÜM.a, . . . , ÜM.d, the signal processing unit 5 selects in the library 12 a standardized reference signal segment Sig_{Hz,kar,ÜM.a}, . . . , Sig_{Hz,kar,ÜM.d} and uses it as an estimated signal segment Sig_{Hz,kar,ÜM(y)}, Sig_{Hz,kar,ÜM(z)}, . . . . The signal processing unit 5 combines the selected estimated signal segments Sig_{Hz,kar,ÜM} with the use of the detected heartbeat times H_Zp(1), H_Zp(2), . . . into the estimated cardiogenic signal Sig_{kar,est}. The signal processing unit preferably interpolates two estimated signal segments located adjacent in time in the signal Sig_{kar,est} in order to fill a gap.

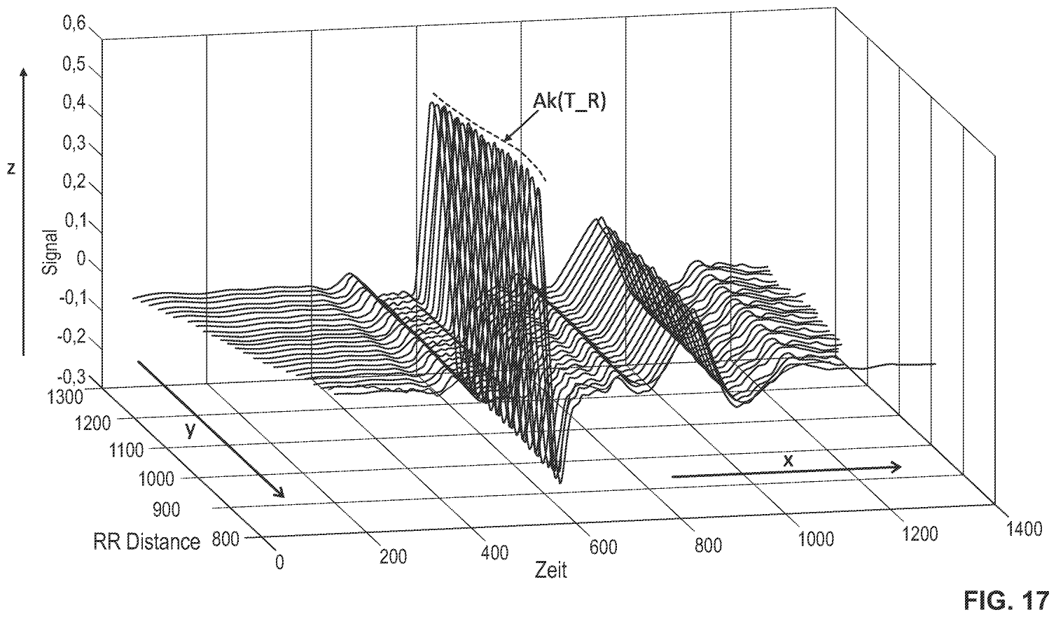

In a number of the versions just shown each sample element comprises a respective sum signal segment or a processed sum signal segment. Depending on the calculated value or calculated values of the transmission channel parameters used, the signal processing unit 5 combines in the training phase Tp the sample elements into classes. The signal processing unit 5 generates for each class a respective reference signal segment, e.g., the four reference signal segments Sig_{Hz,kar,LF.1}, . . . , Sig_{Hz,kar,LF.4} or Sig_{Hz,kar,ÜM.a}, . . . , SigSig_{Hz,kar,ÜM.d}. Different processes are possible for combining the sum signal segments of a class of sample elements into a reference signal segment, which will then be stored in the library 12. FIG. 17 shows such a process as an example.

Time, more precisely, a plurality of relative scanning times, are plotted om the x axis. “Relative” means relative to the beginning of the signal segment. The transmission channel parameter used or a transmission channel parameter used, in this example the R-R interval RR between the R peaks of two consecutive heartbeats, is plotted on the y axis. This process can just as well be used for other transmission channel parameters with two numbers as the parameter values and also for a plurality of transmission channel parameters. The value range of the transmission channel parameter plotted on the y axis is divided in this example into more than ten classes, and in the extreme case up to the accuracy of the machine, i.e., one class per number that can be displayed on the signal processing unit 5 used. The signal value, i.e., the value of the sum signal at this scanning time and at this transmission channel parameter value is plotted on the z axis. The sum signal segments of the sample elements were standardized in advance, so that the standardized sum signal segments Abs_std.x, Abs_std.y have all the same length and the R peaks have the same relative scanning time. These sum signal segments are represented one on top of another with the correct time in the view shown in FIG. 17. All R peaks are located at the relative scanning time T_R.

The signal processing unit 5 calculates in the training phase Tp a fitted curve, which extends in the y-z plane, for each scanning time (x axis) by smoothing. This is illustrated in FIG. 17 for the relative scanning time T_R for the R peak. The signal values which the standardized sum signal segments assume at this scanning time T R yield a point cloud in the y-z plane at the x value T_R. The signal processing unit 5 generates by smoothing over this point cloud a fitted curve, e.g., the fitted curve Ak(T_R) for the scanning time T_R. This is carried out for each scanning time. As a result, a sequence of fitted curves is generated along the x axis. The signal processing unit 5 receives or calculates in the use phase Np the respective value of the transmission channel parameter or each transmission channel parameter for each detected heartbeat at this heartbeat. The transmission channel parameter is an R-R interval in the example shown in FIG. 17. The signal processing unit 5 determines the corresponding class, into which the transmission channel parameter value falls. Each possible transmission channel parameter value forms a class of its own in the extreme case (precision of the machine). The signal processing unit 5 determines for each relative scanning time in the course of this heartbeat the value which the fitted curve, which is associated with this relative scanning time, assumes in this class. This determination yields a signal value. The sequence of the signal values for this class and for the sequence of scanning times is used as the estimated signal segment for this detected heartbeat. Geometrically speaking, the corresponding class specifies a plane, which is at right angles to they axis. The points of intersections of the fitted curve with this perpendicular plane yield the estimated signal segment.

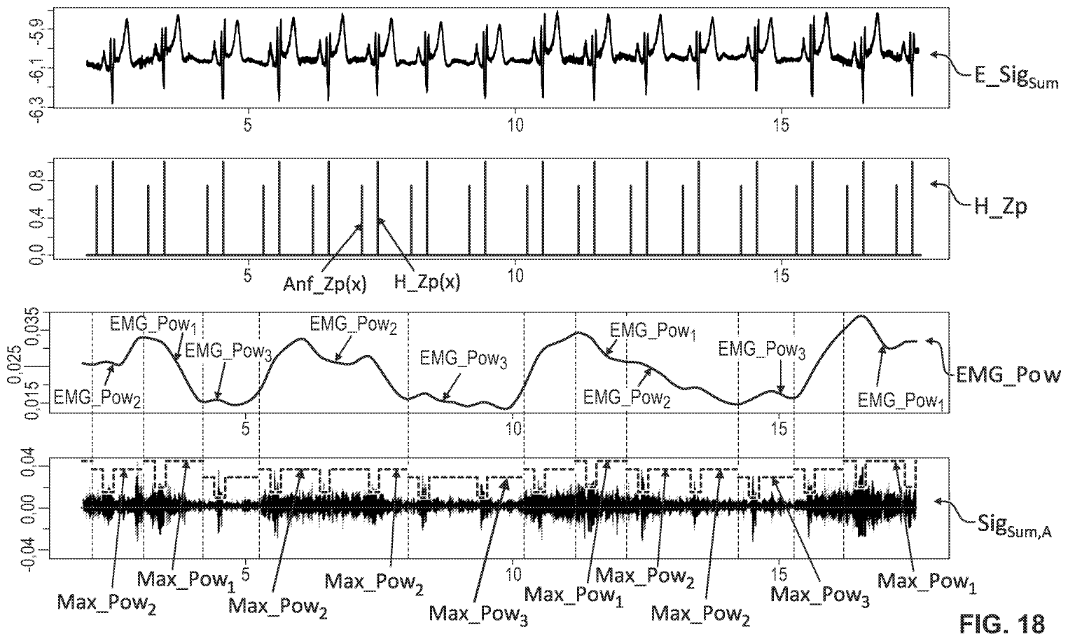

FIG. 18 through FIG. 23 show another variant, in which the cardiogenic signal is determined from a sum signal and a wavelet transformation is applied.

The time curve of the input signal E_Sig_{Sum}, which is generated from electrical measured values of the measuring electrodes 2.1 and 2.2 and results from a superimposition of the heartbeat activity and the breathing activity of the patient P, is shown in the topmost row in FIG. 18. The measured value in mV is plotted on they axis. The sum signal Sig_{Sum} can be generated from this by a corresponding measured value processing.

In the row H_Zp under the input signal E_Sig_{Sum}, the respective beginning, on the one hand, as well as the respective QRS segment of each heartbeat, for example, the beginning Anf_Zp(x) and the QRS segment H_Zp(x) of the xth heartbeat, are shown. The respective QRS segment acts in one embodiment as the characteristic heartbeat time.

The sum signal Sig_{Sum} is subjected to a wavelet transformation, and different frequency ranges are predefined. The wavelet transformation yields a respective signal component for each predefined frequency range. Three signal components A through C are calculated in the example being shown, and more than three signal components are preferably calculated. A respective other process, which will be described below, is carried out for each signal component A through C.

The EMG power (power of the respiratory signal), which is illustrated in FIG. 18, is used as the transmission channel parameter for the signal component A. The influence of the cardiogenic signal Sigₖₐᵣ is compensated for this by calculation in the sum signal Sig_{Sum}, for which purpose, for example, a standard signal segment (standard template) is used, which is valid for each heartbeat, or one of the variants described farther above is used. The compensation yields an estimated respiratory signal Sig_{res,est}, which may still have a relatively great deviation from the actual respiratory signal Sigᵣₑₛ. An envelope, which has exclusively positive signal values, is calculated from the estimated respiratory signal, for example, by calculation of the effective value (root mean square). For example, three classes EMG_Pow₁ (low), EMG_Pow₂ (medium) and EMG_Pow₃ (high) of EMG powers are distinguished. The third row EMG_Pow shows in which segments the current EMG power belongs to which of these three classes.

A respective limit each is determined for each class in the training phase, i.e., a total of three limits Max_Pow₁ (for EMG_Pow₁), Max_Pow₂ (for EMG_Pow₂) and Max_Pow₃ (for EMG_Pow₃) are determined. The row shows the application in the use phase. The cardiogenic component in the signal component A shall be determined. Designated by Sig_{Sum,A} in the signal component A, the values whose respective absolute value is above the respective limit Max_Pow₁, Max_Pow₂, Max_Pow₃ are used as values belonging to the cardiogenic component. Which threshold value it is depends on the current EMG power. The other signal values are set at zero by calculation.

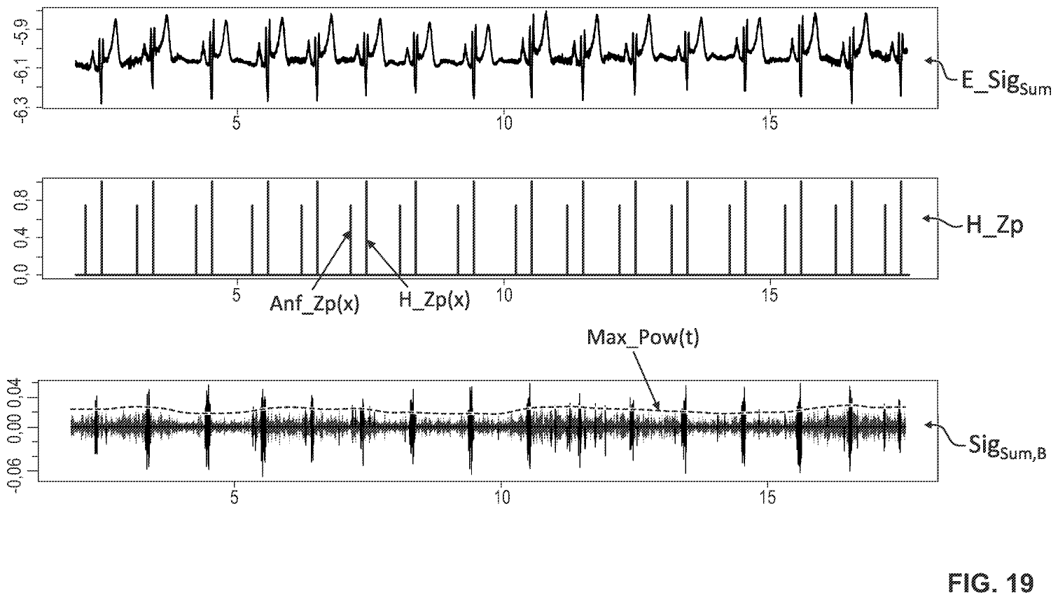

FIG. 19 shows the approach for the signal value B, which is designated by Sig_{Sum,B}. The approach likewise uses the EMG power and differs from the approach for the signal component A as follows: Instead of forming a plurality of classes of EMG powers and then determining a limit for each class, a limit Max_Pow(t), which is variable over time, is calculated. To use the cardiogenic component in the signal component B, a signal value Sig_{Sum,B}(t) above the limit Max_Pow(t) is used for this time t.

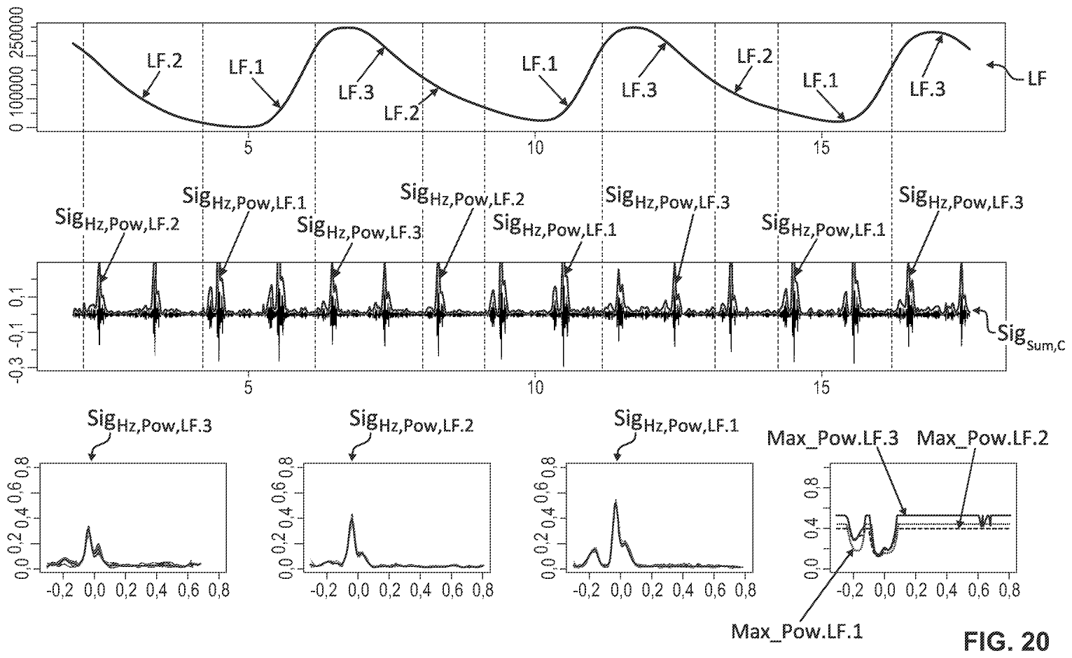

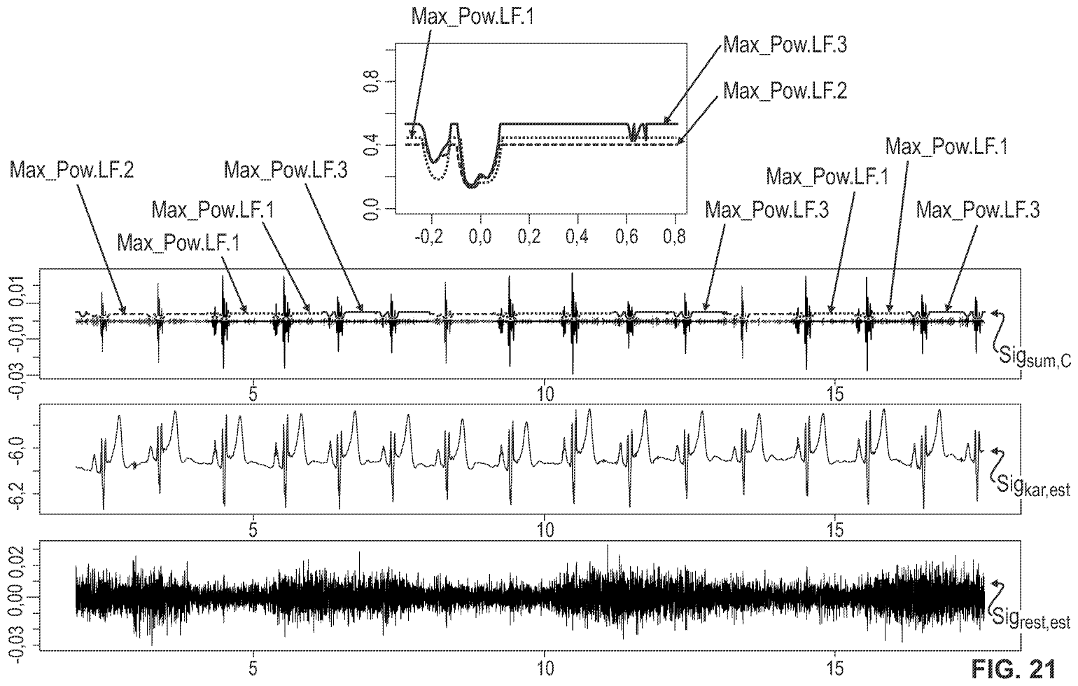

FIG. 20 and FIG. 21 show an approach for the signal component C, which is designated by Sig_{Sum,C}. The lung filling level LF is used as the transmission channel parameter. Three classes of lung filling levels, namely, LF.1, LF.2 and LF.3, are used in this example. The time course of the lung filling level and the respective class are shown in the upper row of FIG. 20. A respective smoothed envelope Sig_{Sum,LF.n} is shown in the middle row of FIG. 20 for each heartbeat depending on the respective class LF.n.

The signal power is calculated from the signal component, e.g., by calculating the effective value (root mean square). This calculation yields a time curve of the signal power. A respective power curve segment is calculated for each heartbeat. Depending on the lung filling level LF.1 or LF.2 or LF.3 at this heartbeat, a power curve segment Sig_{Hz,Pow.LF.1} or Sig_{Hz,Pow.LF.2} or Sig_{Hz,Pow.LF.3} is calculated hereby at this heartbeat.

The power curve segments for a lung filling level class LF.12 or LF.2 or LF.3 are placed one on top of another with the correct time. The segments of one class placed one on top of another are combined, for example, averaged. As a result, a standard power curve segment is formed for each class. The three standard power curve segments Sig_{Hz,Pow,LF.1} and Sig_{Hz,Pow,LF.2} and Sig_{Hz,Pow,LF.3} calculated in this manner are shown in the lower row of FIG. 20. Three limits Max_Pow.LF.1, Max_Pow.LF.2 and Max_Pow.LF.3, which are variable over time, are calculated from these three standard power curve segments for the three classes LF.1, LF.2, LF.3. In one embodiment, the standard power curve segment of one class is scaled and bracketed, for example, by the median of the standard power curve segment being calculated: Median_Pow.LF.n=median(Sig_{Hz,Pow,LF.n}).

The limit Max_Pow.LF.n is then calculated depending on this median, for example, according to the formula

Max Pow.LF.n=min(α*Median Pow.LF.n,β+γ*Median Pow LF.x/Sig_{Hz,Pow,LF.n}).

Where, α, β and γ are predefined constants, for example, α=6, β=0.01 and γ=0.05.

These limits Max_Pow.LF.1, Max_Pow.LF.2 and Max_Pow.LF.3 are the result of the training phase Tp in this approach.

Only the values of the signal component C that belong to the cardiogenic signal, which are above the limit for the respective lung filling level class, are used again in the use phase Np. FIG. 21 again shows, in the upper row, the three limits for the three classes of lung filling level. The signal component C, likewise designated by Sig_{Sum,C}, is shown in the second row.

Depending on the respective lung filling level class LF.1 or LF.2 or LF.3, the respective limit Max_Pow.LF.1 or Max_Pow.LF.2 or Max_Pow.LF.3 is entered.

The respective cardiogenic component in the three signal components A, B and C are combined into an estimated cardiogenic signal Sig_{kar,est}. This estimated cardiogenic signal Sig_{kar,est} is shown in the third row. The difference from the sum signal Sig_{Sum} and from the estimated cardiogenic signal Sig_{kar,est} yields the estimated respiratory signal Sig_{res,est}, which is shown in the fourth row.

It is possible to use an additional transmission channel parameter, namely, the instantaneous EMG power, as this was explained for the signal component B in reference to FIG. 19.

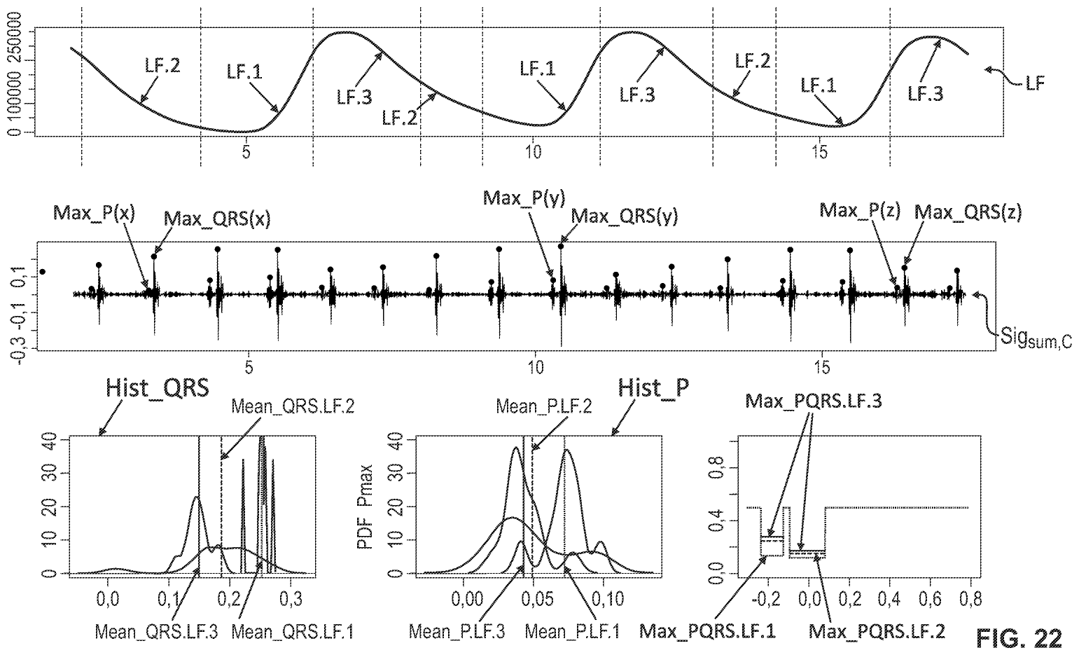

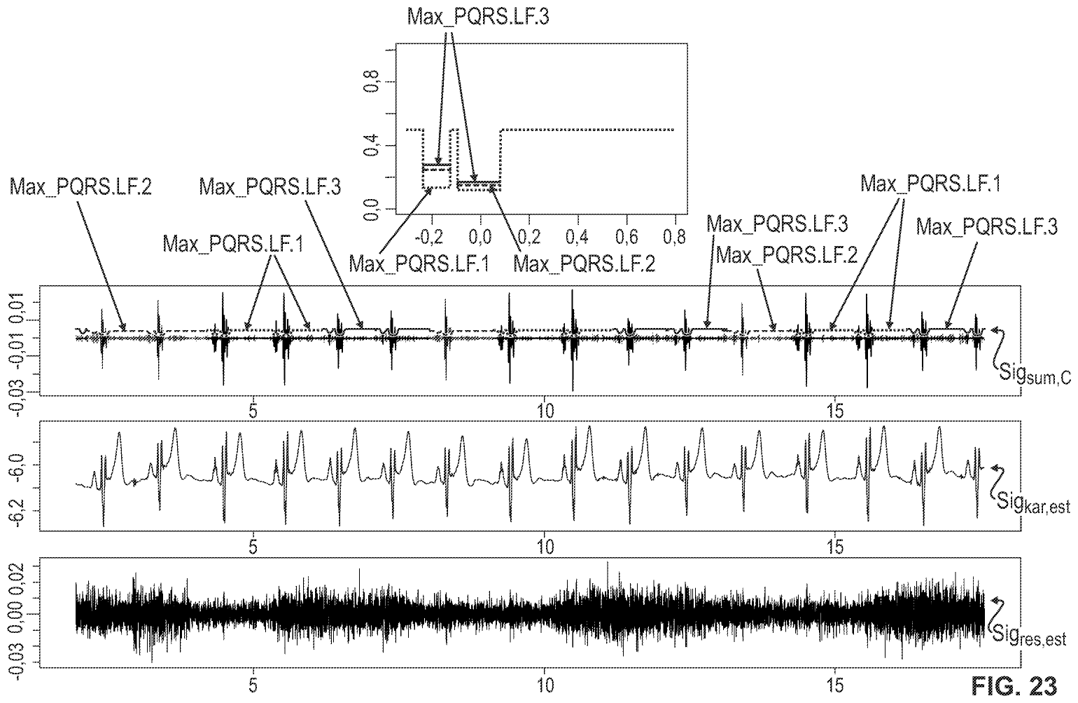

FIG. 22 (training phase) and FIG. 23 (use phase) show a variant of the process for the signal component C. The lung filling level LF is likewise used again as the transmission channel parameter, and three different classes LF.1, LF.2, LF.3 of lung filling levels are likewise distinguished. The time course of these classes LF.1, LF.2, LF.3 is illustrated in the topmost row of FIG. 22.

Two characteristic heartbeat times, namely, the maximum value of the P peak and the maximum value of the QRS area, are detected for each heartbeat in the signal component C, likewise designated by Sig_{Sum,C}. These terms were explained with reference to FIG. 7. Three maximum P values Max_P(x), Max_P(y) and Max_P(z) as well as three maximum QRS values Max_QRS(x), Max_QRS(y) and Max_QRS(z) for three heartbeats x, y, z are shown as an example in FIG. 22.

Two histograms, namely, a histogram Hist_P for the maximum P values and a histogram Hist_QRS for the maximum QRS values, are calculated from these maximum values. The signal value is shown on the x axis and the percentage frequency on the y axis.

Using these two histograms Hist_P and Hist_QRS, three limits, which are variable over time, are again calculated for the three classes LF.1, LF.2, LF.3. These limits are designated by Max_PQRS.LF.1, Max_PQRS.LF.2 and Max_PQRS.LF.3.

A mean value Mean_QRS.LF.x for the class LF.n is calculated by averaging over all maxima Max_QRS(x) of the QRS segments of all heartbeats, which belong to the class LF.n, arithmetically or in another manner. A mean value Mean_P.LF.x, in which averaging is carried out over all maxima Max_P(x) of the P peaks of all heartbeats, which belong to the class LF.n, is correspondingly calculated for the class LF.n. These six mean values are shown in FIG. 22.

A predefined limit is used at the beginning of the use phase Np. As soon as a sufficient number of heartbeats are detected, two different limits are used for each class LF.1, LF.2, LF.3, namely,
a limit according to the calculation rule in the time range of the P-wave of a heartbeat

α1−β1*Mean_P.LF.x and
a limit according to the calculation rule

α2−β2*Mean_QRS.LF.x
in the time range of the QRS segment of a heartbeat.

The four predefined constants have, for example, the values α1=0.05, β1=0.5, α2=0.025 and β2=0.05.

FIG. 23 shows again how the three limits Max_PQRS.LF.1, Max_PQRS.LF.2 and Max_PQRS.LF.3, which are variable over time, are used in order to calculate the estimated cardiogenic signal Sig_{kar,est} and then the estimated respiratory signal Sig_{res,est}.

While specific embodiments of the invention have been shown and described in detail to illustrate the application of the principles of the invention, it will be understood that the invention may be embodied otherwise without departing from such principles.

### LIST OF REFERENCE CHARACTERS

1 Ventilator; it assists the breathing activity of the patient P; it comprises the signal processing unit 5
2.1 Set of measuring electrodes located close to the heart and at a distance from the diaphragm on the chest of the patient P; it acts as a set of sum signal sensors
2.2 Set of measuring electrodes located close to the heart and at a distance from the diaphragm on the abdomen of the patient P; it acts as a set of sum signal sensors
3 Pressure sensor in front of the mouth of the patient P; it acts as a set of sum signal sensors
4 Video camera, which is directed towards the thoracic area of the patient P; it generates the measured value series MWR
5 Signal processing unit; it generates from the sum signal; Sig_{Sum} the estimated respiratory signal Sig_{res,est} and/or the estimated cardiogenic signal Sig_{kar,est}; it comprises the signal processor 13, the heartbeat time detector 7, the reconstructing unit 8 and the compensating unit 9
6 Signal estimating unit; it yields the shape parameter value or each shape parameter value and the expected course Sig_{Hz,kar.LF} of the cardiogenic signal or the expected course of the respiratory signal Sig_{Hz,res.LF} in the course of a single heartbeat depending on the measured values of the transmission channel parameter value or of each transmission channel parameter value; it has reading access to the library 12
7 Heartbeat time detector in the signal processing unit 5; it detects the respective time H_Zp(n) of each heartbeat
8 Reconstructing unit in the signal processing unit 5; it combines the estimated signal segments Sig_{Hz,kar} into the reconstructed (estimated) cardiogenic signal Sig_{kar,est}
9 Compensating unit; it compensates by calculation the influence of the respiratory signal Sigᵣₑₛ on the sum signal Sig_{Sum}
10 Mechanical sensor, which measures a value for the position Pos
11 Heartbeat time period detector; it measures the time period between the two characteristic times H_Zp(x), H_Zp(x+1) of two consecutive heartbeats and/or measures the respective heartbeat time period H_Zr(x), H_Zr(x+1) of each heartbeat
12 Library with an estimated signal segment Sig_{Hz,kar.LF} per class, which describes the estimated cardiogenic signal Sig_{Hz,kar.LF.1}, . . . in the course of a respective heartbeat each
13 Signal processor; it processes the electrical signals from the measuring electrodes 2.1 and 2.2 and/or from the pneumatic sensor 3 and/or from the optical sensor 4; it comprises an amplifier and an analog-digital converter; it performs a baseline removing in one embodiment
14 Sample with sample elements, which are classified according to the transmission channel parameter and comprise each a signal segment in the course of a heartbeat
16 Sensor in the esophagus Sp
Abs.w, Abs.x, Segment of the sum signal Sig_{Sum} in the course of the heartbeat with the
Abs.y, Abs.z characteristic time H_Zp(w) and H_Zp(x) and H_Zp(y) and H_Zp(z)
Abs_DT.w, Processed sum signal segment generated by detrending
Abs_DT.x,
Abs_DT.y
Abs_std.x, Corrected signal segments for a respective heartbeat each; they are all of
Abs std.y equal length and are aligned with the correct time
Ak(T) Fitting curve for the relative scanning time T
AM Breathing muscles of the patient P; they are a source of the respiratory signal Sigᵣₑₛ
Ap Adaptation phase, in which the signal estimating unit 6 is adapted to the current sample elements; overlapped with the use phase Np
Atm.1, Atm.2, . . . Oscillations caused by the breathing activity of the patient P in the estimated respiratory signal Sig_{res,est}
FP-W(1), FP—Set of shape parameter values for a heartbeat
W(2), . . .
H_Zp(n) Time of the nth heartbeat (n=1, 2, . . . ) detected by the heartbeat time detector 7
Hist_P Histogram for the maxima of the P peaks
Hist_QRS Histogram for the maxima of the QRS peaks
HM Heart muscle of the patient P; it is the source of the cardiogenic signal Sigₖₐᵣ
H_Zp(x) Characteristic heartbeat time of the xth heartbeat
H_Zr(x) Heartbeat time period of the xth heartbeat
LF Current lung filling level of the patient P; it is correlated with the volume flow Vol′; it is a transmission channel parameter
LF.1, . . . , LF.4 Classes of lung filling levels, to which a respective reference signal segment Sig_{Hz,kar.LF.1}, . . . , Sig_{Hz,kar.LF.4} each is assigned in one embodiment in the library 12 and to which a set of shape parameter values are assigned in another embodiment; each class is used to estimate the cardiogenic signal Sig_{Hz,kar.LF} or the respiratory signal Sig_{Hz,res.LF} in the course of an individual heartbeat
LQ.a, LQ.b, LQ.c, Exemplary division into classes: It comprises three classes for the lung
Q.d filling level LF and one class for the event that the exhalation time is before the Q wave
Max.1, . . . Maximum, which occurs in the course of a heartbeat in the signal component Sig_{Sum,high} for the higher frequency range
MWR Measured value series with an image sequence, which is recorded by the video camera 4; it yields in one variant the sum signal used
Max_MW.LF.1, Averaged maxima of all segments of the signal component Sigslim.hieh,
Max_MW.LF.2 which belong to the lung filling level LF.1, LF.2, . . . ; stored in the library
Max_P(x) Maximum of the P peak of the xth heartbeat
Mean_P.LF.n Mean value over all maxima Max_P(x) of the heartbeats, at which the lung filling level belongs to class LF.n
Max_Pow.LF.1, Limits for detecting in the signal component C (Sig_{Sum,C}) the cardiogenic
Max_Pow.LF.2, component; they are calculated in the use phase Np depending on the
Max_Pow.LF.3 respective EMP power for the three classes LF.1, LF.2, LF.3
Max_PQRS.LF.1 Limits for the three classes LF.1, LF.2, LF.3 for detecting the cardiogenic
Max_PQRS.LF.2 component in the signal component C (Sig_{Sum.C}); they are calculated
Max_PQRS.LF.3 during the use phase Np as a function of the two histograms Hist_QRS and Hist_P
Max QRS(x) Maximum of the QRS segment of the xth heartbeat
Mean_QRS.LF.n Mean value of the all maxima Max_QRS(x) of the heartbeats at which the lung filling level belongs to class LF.n
Np Use phase; it follows the training phase Tp; it overlaps with the adaptation phase Ap
Npf Useful path; it describes the steps and components during the use phase Np
P Patient, whose intrinsic breathing activity is assisted by the ventilator 1; he is measured by the measuring electrodes 2.1 and 2.2, by the pneumatic sensor 3 and by the video camera 4
Pos Position of a measuring electrode 2.1, 2.2 relative to the heart of the patient P, measured by sensor 10; it acts as an additional transmission channel parameter
Sig_{ges} Overall signal for the breathing and ventilation of the patient P; it is generated by a superimposition of the intrinsic breathing activity of the patient P and the mechanical ventilation by the ventilator 1
Sig_{kar,est} Reconstructed (estimated) cardiogenic signal, combined from the estimated cardiogenic signal segments Sig_{Hz,kar} with the use of the heartbeat times H_Zp(n)
Sig_{Hz,kar} Estimated signal segment: Segment of the cardiogenic signal in the course of an individual heartbeat, provided by the signal estimating unit 6
Sig_{Hz,kar.LF} Estimated cardiogenic signal segment; it is the segment of the estimated cardiogenic signal Sig_{kar,est} in the course of a single heartbeat which is adapted to the current value LF.1, . . . , LF.4 of the transmission channel parameter or each transmission channel parameter (here: lung filling level LF); provided by the signal estimating unit 6
Sig_{Hz,kar,LF.1, . . . Cardiogenic reference signal segments stored in teeth library 12 for the}
Sig_{Hz,kar,LF.4} four classes LF.1, . . . , LF.4 of the lung filling level LF
Sig_{Hz,kar,ÛM.a}, Cardiogenic reference signal segments stored in the library 12 for the four classes ÜM.a, . . . , ÜM.d of agreement values with the reference
Sig_{Hz,kar,ÜM.d} curves V.1, V.2
Sig_{Hz,kar,ÜM Estimated cardiogenic signal segment, provided by the signal estimating unit 6 as a function of the overall agreement value}
Sigₖₐᵣ Cardiogenic signal; it describes the cardiac activity of the patient P
Sig_{kar,est} Estimate for the cardiogenic signal Sigₖₐᵣ, generated by the signal processing unit 5
Sig_{Hz,Ref} Predefined standard reference signal segment, average cardiogenic signal segment in the course of an individual heartbeat
Sigᵣₑₛ Respiratory signal; it describes the intrinsic breathing activity of the patient P
Sig_{res,est} Estimate generated by the signal processing unit 5 for the respiratory signal Sigᵣₑₛ
Sig_{Hz,res,LF} Estimated respiratory signal segment, the segment of the estimated respiratory signal in the course of an individual heartbeat, which is adapted to the current value LF.1, . . . , LF.4 of the transmission channel parameter or of each transmission channel parameter (here: lung filling level LF, provided by the signal estimating unit 6 as a function of at least one transmission channel parameter value
Sig_{Hz,res,LF.1}, . . . , Respiratory reference signal segments stored in the library 12 for the four
Sig_{Hz,res,LF.4} classes LF.1, LF.4 of the lung filling level LF
Sig_{Sum} Sum signal, measured by the sum signal sensors 2.1, 2.2, 3 or 4; it is a superimposition of the respiratory signal Sigᵣₑₛ and of the cardiogenic signal Sigₖₐᵣ
Sig_{Sum,high} Component in the sum signal Sig_{Sum}, which is in the higher frequency range
Sig_{Sum,low} Component in the sum signal Sig_{Sum}, which is in the lower frequency range

S_Q Signal, which describes another transmission channel parameter, namely, whether the exhalation by the patient begins shortly before the Q wave or not
Sp Esophagus of the patient P
Tnn Additional transmission channel for the cardiogenic signal Sigₖₐᵣ; it begins in the heart muscle
Tns Transmission channel for the cardiogenic signal Sigₖₐᵣ; it leads from the heart muscle to the sensor 2.1, 2.2
Tss Transmission channel for the respiratory signal Sigᵣₑs; it leads from the breathing muscles to the sensor 2.1, 2.2
Tp Training phase; it precedes the adaptation phase Ap
Tpf Training path; it describes the steps and components during the training phase Tp and the subsequent adaptation phase Ap
T_R Relative scanning time, on which the R peak falls
ÜM.1, ÜM.2, . . . Overall agreement value; it depends on the agreement between a sum signal segment and a reference curve V.1, V.2
Vol′ Volume flow of breathing air into and out of the airway Aw; it is correlated with the lung filling level LF, it is a transmission channel parameter, which is correlated with an anthropological size (hcrc: lung filling level LF), which influences the transmission channel Tns
V.1, . . . , V.3 Reference curves, generated by singular value decomposition (SVD) from the standardized sum signal segments Abs_std.x, Abs_std.y, . . .
Zw Diaphragm of the patient P

1. A computer-implemented process for calculating an estimate for a cardiogenic signal and/or a respiratory signal with the use of a signal processing unit, wherein the cardiogenic signal is an indicator for a cardiac activity of a patient and the respiratory signal is an indicator for a for an intrinsic spontaneous breathing and/or a mechanical ventilation of the patient, wherein the process comprises a training phase and a subsequent use phase, the process further comprising the steps of:
receiving and processing, with the signal processing unit, at least during the training phase measured values from a sum signal sensor device, which sensor device measures a signal generated in the body of the patient;
generating, with the signal processing unit at least in the training phase, depending on a time course of measured values of the sum signal sensor device, a sum signal, which comprises a superimposition of the cardiac activity and the intrinsic spontaneous breathing and/or mechanical ventilation of the patient;
detecting during the training phase, with the signal processing unit, a plurality of heartbeats, which the patient performs during the training phase;
generating during the training phase, with the signal processing unit, a sample with a plurality of sample elements, wherein each sample element pertains to a respective detected heartbeat,
wherein the generation of a sample element for a heartbeat comprises the steps of:
determining, with the signal processing unit, a sum signal segment of the sum signal, which sum signal segment pertains to the heartbeat;
determining, with the signal processing unit, for at least one shape parameter a shape parameter value which the shape parameter assumes during this the heartbeat by analysis of the sum signal segment, wherein the shape parameter influences a time course of the cardiogenic signal and/or of the respiratory signal;
receiving, with the signal processing unit, at least one value of a predefined first transmission channel parameter, which value has been measured during the heartbeat by an additional sensor, or calculating, with the signal processing unit, the value of the first transmission channel parameter by an analysis of the sum signal, wherein the first transmission channel parameter correlates with an effect of an anthropological variable on a transmission channel from a signal source in the body of the patient to the sum signal sensor device; and
generating, with the signal processing unit, the sample element for the heartbeat such that the sample element comprises the shape parameter value calculated for the heartbeat and the value of the first transmission channel parameter measured or calculated for the heartbeat;
generating, with the signal processing unit during the training phase, with the use of the sample, a signal estimating unit, which unit yields the shape parameter as a function of the first transmission channel parameter;
detecting, with the signal processing unit, during the use phase at least one heartbeat which the patient performs during the use phase;
during the use phase carrying out the following steps for at least one detected heartbeat:
detecting, with the signal processing unit, a characteristic heartbeat time or a heartbeat time period of the heartbeat;
receiving a value of the first transmission channel parameter, which value has been measured at the heartbeat, from the additional sensor or generating the sum signal also during the use phase depending on measured values of the sum signal sensor device and calculating a value of the first transmission channel parameter for the heartbeat by an analysis of the sum signal;
calculating, a shape parameter value for the shape parameter by applying the signal estimating unit to the value of the first transmission channel parameter measured or calculated for the heartbeat; and
calculating, with the use of the calculated shape parameter value, an estimated cardiogenic signal segment and/or an estimated respiratory signal segment for the heartbeat, which segment approximately describes the cardiogenic signal or the respiratory signal, respectively, during the heartbeat;
carrying out, with the signal processing unit, at least one of the following three steps during the use phase with the use of the characteristic heartbeat time or the heartbeat time period detected during the use phase;
combining the calculated estimated cardiogenic signal segments for the detected heartbeats to the estimated cardiogenic signal; or
combining the calculated estimated respiratory signal segments for the detected heartbeats to the estimated respiratory signal or
determining the estimated respiratory signal by compensating the cardiac activity by calculation, wherein the step of determining during the use phase the estimated respiratory signal by compensation comprises the following steps with the signal processing unit:
generating a sum signal during the use phase depending on measured values of the sum signal sensor device and
compensating an influence of the heartbeat on the sum signal generated during the use phase, the compensation is performed by using the estimated cardiogenic signal segment for the heartbeat.

2. A process in accordance with claim 1,
wherein the step of generating the signal estimating unit with the use of the sample comprises the steps performed by the signal processing unit of
splitting up the sample elements on the basis of the respective values of the first transmission channel parameter into sample element classes such that the values of the first transmission channel parameter of the sample elements of one sample element class differ from one another by at most one predefined absolute or percentage limit, and
calculating for each sample element class a respective reference value range for the first transmission channel parameter and an associated reference signal segment, wherein the signal processing unit combines the sum signal segments of the sample element class to the reference signal segment and wherein the associated reference signal segment acts as the shape parameter, and wherein the signal processing unit generates the signal estimating unit such that the signal estimating unit comprises a library with a plurality of reference signal segments, wherein each reference signal segment is assigned to a reference value range of the first transmission channel parameter, and
wherein the step of applying in the use phase the signal estimating unit to a value of the first transmission channel parameter comprises the steps performed by the signal processing unit of
determining, depending on the received value of the first transmission channel parameter, at least one reference value range of the first transmission channel parameter and the respective associated reference signal segment and
calculating the estimated signal segment depending on the determined reference signal segment.

3. A process in accordance with claim 2,
wherein the signal processing unit calculates during the training phase for each sample element class a respective reference value of the first transmission channel parameter by using the values of the first transmission channel parameter belonging to the sample element class and uses the respective reference value of the first transmission channel parameter as the value range of the first reference transmission channel parameter of the sample element class and
wherein the step of applying in the use phase the signal estimating unit to the value of the first transmission channel parameter measured at a heartbeat comprises the steps performed by the signal processing unit of
determining in the library a first and a second reference signal segment, which are associated with a first and a second reference value of the first transmission channel parameter as the respective value range of the first transmission channel parameter of the first and second reference signal segment, wherein the first reference transmission channel parameter value is lower than or equal to and the second reference transmission channel parameter value is greater than or equal to the value of the first transmission channel parameter measured at the heartbeat, and
calculating the signal segment estimated for the heartbeat by a smoothing between the first determined reference signal segment and the second determined reference signal segment.

4. A process in accordance with claim 2,
wherein at least one reference time course of the sum signal during a heartbeat is predefined or is calculated by the signal processing unit during the training phase and
wherein in the step of receiving or calculating for a heartbeat a value of the first transmission channel parameter, the signal processing unit
determines the sum signal segment belonging to the heartbeat,
calculates a respective agreement value between this sum signal segment and the reference time course and
calculates the value of the first transmission channel parameter for the heartbeat with the use of the calculated agreement value, wherein each class of sample elements, which the signal processing unit generates during the use phase, comprises as the reference value of the first transmission channel parameter value range a respective value range of possible agreement values.

5. A process in accordance with claim 4, wherein the signal processing unit calculates during the training phase the reference time courses with the use of the sum signal segments determined in the training phase by applying a singular value decomposition or a principal component analysis to predefined standardized sum signal segments.

6. A process in accordance with claim 2, wherein the step of the signal processing unit combining, for a sample element class, the sum signal segments of the class to the reference signal segment comprises the steps performed by the signal processing unit of
superimposing the sum signal segments of the sample element class by calculation, so that each sum signal segment pertains to the same sequence of relative sampling time points,
generating, for each relative sampling time point by applying a smoothing procedure, a respective fitting curve, which assigns a respective reference signal value to each value range of the first transmission channel parameter belonging to a sample element class, and
determining for each value range of the first transmission channel parameter a sequence of the fitting curve values along the relative sampling time points and using the sequence as the reference signal segment for the value range of the first transmission channel parameter.

7. A process in accordance with claim 1, wherein the step of compensating, by calculation, the influence of the heartbeat on the sum signal during the determination of the estimated respiratory signal comprises the steps performed by the signal processing unit of
determining a heartbeat time period of the heartbeat and
compensating, by calculation, and by the use of the cardiogenic signal segment estimated for the heartbeat, the influence of the heartbeat on the segment of the sum signal that pertains to the heartbeat time period.

8. A process in accordance with claim 1,
wherein the generation of the sample element for a heartbeat comprises the additional steps that the signal processing unit
receives a value of at least one additional predefined transmission channel parameter, which value was measured in the course of the heartbeat and wherein the additional transmission channel parameter correlates with an effect of the anthropological variable or of another anthropological variable on the first transmission channel or on an additional transmission channel guiding to the sum signal sensor device and
generates the sample element for the heartbeat such that the sample element additionally comprises the value of the additional transmission channel parameter, which value was measured in the course of the heartbeat, the signal processing unit generates the signal estimating unit such that the signal estimating unit yields for a heartbeat the shape parameter as a function of the first transmission channel parameter and of the additional transmission channel parameter, and
wherein the signal processing unit carries out in the use phase for at least one detected heartbeat the additional steps that the signal processing unit
receives the respective measured value from the additional sensor or calculates same by an analysis of the sum signal, which measured or calculated values the first transmission channel parameter and the additional transmission channel parameter, respectively, assume at the heartbeat, and
calculates a respective value for the shape parameter by applying the signal estimating unit to the respective value of the first and the additional transmission channel parameter measured or calculated at the heartbeat.

9. A process in accordance with claim 8, wherein the first transmission channel parameter is correlated with a filling level of the lungs of the patient and the additional transmission channel parameter is correlated with a phase during an individual breathing operation and/or ventilating operation.

10. A process in accordance with claim 1, wherein the first transmission channel parameter depends on a geometry of the body of the patient and the signal processing unit receives and processes both in the training phase and in the use phase a plurality of measured values, which values have been measured by a body geometry sensor, wherein the measured values of the body geometry sensor correlate with the body geometry of the patient, which is current.

11. A process in accordance with claim 10, wherein the first transmission channel parameter, is a current breathing state and/or ventilating state of the patient and the body geometry sensor comprises a breathing state sensor, which measures the current breathing state and/or ventilating state of the patient.

12. A process in accordance with claim 11, wherein the breathing state sensor measures at least one of
a flow of gas into the body and/or out of the body of the patient,
an airway pressure of the patient,
a flow of gas out of a mechanical ventilator or into a ventilator, wherein the ventilator is in a fluid connection with the patient and
a current position, speed and/or acceleration of at least one reference point on the skin of the patient.

13. A process in accordance with claim 11,
wherein the sum signal sensor device comprises at least one sum signal sensor positioned on the skin of the patient,
wherein the signal processing unit receives measured values for a current position relative to a reference point of the sum signal sensor positioned on the skin,
wherein the position sensor measures the relative position of the sum signal sensor during both the training phase and the use phase,
wherein during the training phase, the signal processing unit
generates a functional relationship by means of measured values of the breathing state sensor and of measured values of the position sensor, which functional relationship describes the relative position of the sum signal sensor positioned on the skin as a function of the breathing state and/or ventilating state of the patient, and
generates the signal estimating unit such that the signal estimating unit yields for a heartbeat the shape parameter as a function of the measured relative position of the sum signal sensor positioned on the skin, and
wherein during the use phase, for at least one detected heartbeat, the signal processing unit
receives measured values, which correlate with the current breathing state and/or ventilating state of the patient during the heartbeat,
calculates the current relative position of the sum signal sensor by applying the functional relationship to the measured current breathing state and/or ventilating state and
calculates the estimated signal segment for the heartbeat by applying the signal estimating unit to the calculated relative position.

14. A process in accordance with claim 1, wherein the signal processing unit measures the value of the first transmission channel parameter or of an additional transmission channel parameter by the signal processing unit analyzing the received sum signal, which value of the additional transmission channel parameter is measured in the course of the heartbeat and wherein the additional transmission channel parameter correlates with an effect of the anthropological variable or of another anthropological variable on the transmission channel or on an additional transmission channel guiding to the sum signal sensor device.

15. A process in accordance with claim 14, wherein the transmission channel parameter or the additional transmission channel parameter is measured by analysis of the sum signal and is
an interval between two characteristic times of two consecutive heartbeats or
an interval between two signal peaks in a course of a single heartbeat or
an difference between a highest value and a lowest value of the sum signal in the course of a single heartbeat.

16. A process in accordance with claim 1,
wherein a standard reference signal segment, which is caused by the cardiac activity in a course of a heartbeat, is predefined,
wherein this standard reference signal segment depends on the shape parameter,
wherein the generation of the sample element for a heartbeat comprises the steps of the signal processing unit
calculating a value for the shape parameter of the standard reference signal segment by analyzing the sum signal segment belonging to the heartbeat and
generating the sample element for the heartbeat such that the sample element comprises the value of the shape parameter, which value is calculated for the heartbeat,
wherein the signal processing unit generates the signal estimating unit such that the signal estimating unit yields the shape parameter of the standard reference signal segment as a function of the first transmission channel parameter, and
wherein the step of calculating in the use phase the estimated signal segment for a detected heartbeat comprises the steps performed by the signal processing unit of
calculating the value of the shape parameter of the standard reference segment by applying the signal estimating unit to the value of the first transmission channel parameter, which value was measured at a detected heartbeat,
adapting the predefined standard reference signal segment with the use of the calculated value of the shape parameter, and
calculating the estimated signal segment for the heartbeat depending on the adapted standard reference signal segment.

17. A process in accordance with claim 1,
wherein the sum signal sensor device comprises at least one sum signal sensor located at a distance from the heart and at least one sum signal sensor located closer to the heart, wherein both sum signal sensors measure a signal generated in the body of the patient,
wherein the sum signal sensor located at a distance from the heart has a greater distance from a heart muscle of the patient than the sum signal sensor located closer to the heart, and
wherein the signal processing unit
generates the sum signal during the training phase by using measured values of the sum signal sensor located at a distance from the heart and
detects during the use phase the heartbeat and the characteristic time thereof and/or the heartbeat time period of the heartbeat with measured values of the sum signal sensor located closer to the heart.

18. A process in accordance with claim 17,
wherein the sum signal sensor located at a distance from the heart has a shorter distance from a muscle of breathing muscles of the patient than the sum signal sensor located closer to the heart,
wherein during the use phase, the signal processing unit
generates the sum signal with the use of measured values of the sum signal sensor located at a distance from the heart
without using the measured values of the sum signal sensor located closer to the heart.

19. A process in accordance with claim 1,
wherein the sensor signal device comprises at least one first sum signal sensor and at least one second sum signal sensor;
wherein at least during the training phase, the signal processing unit receives
measured values from the first sum signal sensor, which first sum signal sensor measures the signal generated in the body of the patient at a first position, and
measured values from the second sum signal sensor, which second sum signal sensor measures the signal generated in the body of the patient at a second position different from the first position, and
wherein the process comprises the additional steps that during the training phase, the signal processing unit
generates a first sum signal depending on measured values of the first sum signal sensor and
generates a second sum signal depending one measured values of the second sum signal sensor
generates a first sample with the use of the first sum signal and a second sample with the use of the second sum signal and
generates a first signal estimating unit with the use of the first sample and a second signal estimating unit with the use of the second sample and
wherein during the use phase, for at least one detected heartbeat, the signal processing unit
generates a first estimated signal segment for the heartbeat by applying the first signal estimating unit and a second estimated signal segment for the heartbeat by applying the second signal estimating unit and
combines the first and second estimated signal segments into an estimated signal segment for the heartbeat.

20. A process in accordance with claim 19, wherein the generation of the sample element for the detected heartbeat comprises the following steps:
the signal processing unit
receives measured values from a first parameter sensor, which first parameter sensor measures a first value of the first transmission channel parameter, and
receives measured values from a second parameter sensor, which second parameter sensor measures a second value of the first transmission channel parameter or of an additional transmission channel parameter,
during the training phase, the signal processing unit
generates the first signal estimating unit such that the first signal estimating unit yields the shape parameter as a function of the first transmission channel parameter measured by the first parameter sensor, and
generates the second signal estimating unit such that the second signal estimating unit yields the shape parameter as a function of the transmission channel parameter measured by the second parameter sensor, and
in the use phase, for the detected heartbeat, the signal processing unit
receives a first parameter value, which first parameter value was measured by the first parameter sensor during the heartbeat,
receives a second parameter value, which second parameter value was measured by the second parameter sensor during the heartbeat,
generates the first estimated signal segment for the heartbeat by applying the first signal estimating unit to the first parameter value and
generates the second estimated signal segment for the heartbeat by applying the second signal estimating unit to the second parameter value.

21. A process in accordance with claim 1,
wherein during the training phase, the signal processing unit carries out the steps of
generating the sum signal in the time range,
transforming for each heartbeat the segment of the sum signal, belonging to the heartbeat, into a sum signal in the frequency range,
determining the shape parameter value by an analysis of the sum signal segment transformed into the frequency range,
generating each sample element for a heartbeat such that the sample element comprises the shape parameter value determined in the frequency range and the value of the first transmission channel parameter value measured during the heartbeat, and
generating the signal estimating unit such that the signal estimating unit describes in the frequency range the shape parameter as a function of the first transmission channel parameter,
wherein during the use phase, the signal processing unit carries out for at least one detected heartbeat the steps of
calculating an estimated signal segment in the frequency range by applying the signal estimating unit and
transforming the estimated signal segment into an estimated signal segment in the time range.

22. A process in accordance with claim 1,
wherein at least one first frequency range is predefined and the process comprises the additional steps of the signal processing unit
generating from the measured values of the sum signal sensor device and overall sum signal,
determining in the overall sum signal a respective signal component that is in the first frequency range, and
determining in the signal component, which is in the first frequency range, a respiratory signal component and/or a cardiogenic signal component and
wherein the signal processing unit furthermore takes the action of
determining the estimated respiratory signal with the use of the respiratory signal component located in the first frequency range and/or
determining the estimated cardiogenic signal with the use of the cardiogenic signal component in the first frequency range.

23. A process in accordance with claim 22, wherein at least one second frequency range is predefined such that the signal component of the overall sum signal, which signal component is located in the second frequency range, is effected exclusively by the intrinsic spontaneous breathing and/or mechanical ventilation or exclusively by the cardiac activity of the patient wherein the signal processing unit
determines the estimated respiratory signal with the use of the respiratory signal component in the first frequency range and of the signal component of the overall sum signal, which said signal component of the overall sum signal is located in the second frequency range and is effected by the breathing/ventilation or
determines the estimated cardiogenic signal with the use of the cardiogenic signal component in the first frequency range and the, signal component of the overall sum signal, which said signal component of the overall sum signal is located in the second frequency range, and is effected by the cardiac activity.

24. A process in accordance with claim 1,
wherein a change rule is predefined, which is applicable to a segment of the sum signal belonging to one heartbeat,
wherein the predefined change rule depends on the shape parameter and
wherein during the use phase the signal processing unit
calculates a value for the shape parameter in the predefined change rule by applying the signal estimating unit and
in the step of calculating an estimated signal segment for the detected heartbeat, the signal processing unit applies the change rule, which is parameterized with the calculated shape parameter value, to the segment of the sum signal, which segment belongs to the heartbeat, and the signal processing unit calculates the estimated signal segment by applying the parameterized change rule to the sum signal segment.

25. A process in accordance with claim 1,
wherein a subdivision of the heartbeat time period into at least two different heartbeat time period phases, which subdivision is valid for each heartbeat, is predefined,
wherein during the training phase and during the use phase the signal processing unit receives a respective value for the first transmission channel parameter and for each heartbeat time period phase, which value was received from the additional sensor during the heartbeat time period phase,
wherein during the training phase the signal processing unit generates for each detected heartbeat and for each heartbeat time period phase of the detected heartbeat a respective sample element such that the respective sample element comprises the shape parameter value calculated for the heartbeat time period phase and the value of the first transmission channel parameter, which channel parameter value was measured during the heartbeat time period phase, and
wherein during the use phase, for the detected heartbeat, the signal processing unit
calculates for each heartbeat time period phase of the detected heartbeat a respective value for the shape parameter by applying the signal estimating unit to the value of the first transmission channel parameter measured during the heartbeat time period phase and
calculates the estimated signal segment for the heartbeat with the use of the shape parameter values for the heartbeat time period phases of the detected heartbeat.

26. A process in accordance with claim 25,
wherein during the training phase, the signal processing unit generates for each heartbeat time period phase, with the use of the sample elements generated for the heartbeat time period phase, a respective signal phase estimating unit, which yields the shape parameter as a function of the first transmission channel parameter and is valid for the heartbeat time period phase, and
wherein during the use phase, for the detected heartbeat, the signal processing unit
calculates for each heartbeat time period phase of the heartbeat a respective estimated signal segment, which approximately describes the cardiogenic signal or the respiratory signal in the course of the heartbeat time period phase of the heartbeat, and
calculates the estimated signal segment for the heartbeat with the use of the estimated signal segments for the heartbeat time period phases of the heartbeat and uses the signal phase estimating unit for the heartbeat time period phase for this calculation.

27. A process in accordance with claim 1,
wherein during the use phase, the signal processing unit
generates at least one additional sample element which additional sample element relates to a heartbeat detected during the use phase, and
wherein the signal estimating unit generated during the training phase is modified or generated again with the use of the additional sample element generated during the use phase.

28. A process in accordance with claim 1,
wherein the patient is ventilated by means of a mechanical ventilator, which carries out ventilation strokes,
wherein the ventilation strokes are triggered depending on the determined estimated respiratory signal.

29. A process in accordance with claim 1,
wherein the patient is ventilated by means of a mechanical ventilator and
wherein the signal processing unit performs the additional steps in the use phase of
receiving a measured ventilator signal, which describes the flow of gas effected by the ventilator between the ventilator and the patient,
comparing the ventilator signal with the estimated respiratory signal
depending on the comparison result, calculating an assessment of the synchronization between the breathing activity of the patient and the gas flow effected by the ventilator and
if this assessment of the synchronization is below a predefined threshold, causing an operating parameter of the ventilator to change automatically and/or an alarm to be outputted.

30. A signal processing unit for calculating an estimate for a cardiogenic signal and/or a respiratory signal, wherein the cardiogenic signal is an indicator for a cardiac activity of a patient and the respiratory signal is an indicator for an intrinsic spontaneous breathing and/or a mechanical ventilation of the patient,
wherein the signal processing unit is configured for carrying out a training phase and a subsequent use phase,
wherein the signal processing unit is configured to receive, at least during the training phase, measured values from a sum sensor device, which sum sensor device is configured to measure a signal generated in the body of the patient, and the signal processing unit is configured to process these measured values,
wherein the signal processing unit is configured to generate, at least during the training phase, depending on the time course of measured values of the sum signal sensor device, a sum signal, which comprises a superimposition of the cardiac activity and of the intrinsic spontaneous breathing and/or mechanical ventilation of the patient,
wherein the signal processing unit is configured to
detect during the training phase a plurality of heartbeats, which the patient performs during the training phase, and
generate during the training phase a sample with a plurality of sample elements, wherein each sample element pertains to one respective detected heartbeat,
wherein the signal processing unit is configured to carry out the following steps during the generation of a sample element for a heartbeat:
determine a segment of the sum signal, which sum signal segment belongs to the heartbeat,
determine by analyzing the sum signal segment for at least one shape parameter a shape parameter value that the shape parameter assumes at the heartbeat, wherein the shape parameter influences a time course of the cardiogenic signal and/or of the respiratory signal,
receive at least one value of a predefined first transmission channel parameter, which value has been measured at the heartbeat by an additional sensor or
generate a sum signal during the use phase depending on measured values of the sum signal sensor device and to calculate a value of the first transmission channel parameter by analysis of the sum signal, wherein the first transmission channel parameter correlates with an effect of an anthropological variable on a transmission channel from a signal source in the body of the patient to the sum signal sensor device, and
generate the sample element for the heartbeat such that the sample element comprises the shape parameter value and the value of the first transmission channel parameter, which values were measured or calculated at the heartbeat,
wherein the signal processing unit is configured to generate with the use of the sample a signal estimating unit, which unit yields the shape parameter as a function of the first transmission channel parameter,
wherein the signal processing unit is configured to detect during the use phase at least one heartbeat, which the patient performs during the use phase,
wherein the signal processing unit is configured to carry out the following steps for at least one heartbeat detected during the use phase:
detecting a characteristic heartbeat time or a heartbeat time period of the heartbeat,
receiving a value of the first transmission channel parameter, which value was measured during the heartbeat by the additional sensor, or calculating such a value by an analysis of the sum signal,
calculating a value for the shape parameter by applying the signal estimating unit to the value of the first transmission channel parameter, the value being measured or calculated for the heartbeat, and
calculating with the use of the calculated shape parameter value an estimated cardiogenic signal segment and/or an estimated respiratory signal segment for the heartbeat, which signal segment describes the cardiogenic signal or the respiratory signal, respectively, in the course of the,
wherein the signal processing unit is configured to carry out during the use phase at least one of the following three steps, with the use of the characteristic heartbeat time or heartbeat time period detected during the use phase:
combining the calculated estimated cardiogenic signal segments for the detected heartbeats to the estimated cardiogenic signal or
combining the calculated estimated respiratory signal segments for the detected heartbeats to the estimated respiratory signal or
determining the estimated respiratory signal by compensating the cardiac activity,
wherein the signal processing unit is configured to carry out the following steps when determining the estimated respiratory signal by compensating:
generating a sum signal depending on measured values of the sum signal sensor device during the use phase as well and
compensating by calculation the influence of the heartbeat on the sum signal generated during the use phase, wherein the compensation is performed by using the estimated cardiogenic signal segment for the heartbeat.

31. A process according to claim 1, wherein a computer program is provided, which can be executed on the signal processing unit wherein an execution of the program causes, during execution on the signal processing unit when the signal processing unit receives measured values from the sum signal sensor device, the signal processing unit to carry out at least some of the process steps.

32. A process according to claim 1, wherein a signal sequence is provided, comprising commands, which can be executed on the signal processing unit, wherein an execution of the commands on the signal processing unit causes, when the signal processing unit receives measured values from the sum signal sensor device, the signal processing unit to carry out at least some of the process steps.
